(19) United States
(12) Patent Application Publication
(10) Pub. No.: US 2014/0075596 A1
(43) Pub. Date: Mar. 13, 2014

(54) siRNAS Compositions and Method for Manipulating Berry Ripening

(71) Applicant: Texas Tech university System, Lubbock, TX (US)

(72) Inventors: Christopher D. Rock, Lubbock, TX (US); Qingjum Luo, Sunnyvale, CA (US)

(73) Assignee: Texas Tech university System, Lubbock, TX (US)

(21) Appl. No.: 13/874,962

(22) Filed: May 1, 2013

Related U.S. Application Data
(60) Provisional application No. 61/641,045, filed on May 1, 2012.

(51) Int. Cl. C12N 15/113

(52) U.S. Cl. CPC C12N 15/113
USPC 800/285; 435/419; 435/416; 536/23.1

(57) ABSTRACT

The present invention provides a method and compositions of regulating plant development and secondary metabolite biosynthesis by providing one or more plant cells; providing a small interfering RNAs (ta-siRNAs) to the one or more plant cells; targeting a transcription factor to affect the plant development and/or secondary metabolite biosynthesis.

### CROSS-REFERENCE TO RELATED APPLICATIONS

This Non-Provisional patent application claims priority to U.S. Provisional Patent Application Ser. No. 61/641,045, filed May 1, 2012, the contents of which is incorporated by reference herein in its entirety.

### STATEMENT OF FEDERALLY FUNDED RESEARCH

This invention was made with government support under Grant No. 1R21-GM077245-01A1 awarded by the NIH. The government has certain rights in the invention.

### TECHNICAL FIELD OF THE INVENTION

The present invention relates in general to the field of plant ripening, specifically to methods of making and using compositions of matter for manipulating grape berry ripening by small RNAs.

### INCORPORATION-BY-REFERENCE OF MATERIALS FILED ON COMPACT DISC

None

### BACKGROUND OF THE INVENTION

Without limiting the scope of the invention, its background is described in connection with making and using small RNAs compositions for manipulating grape berry ripening. The MYB superfamily constitutes the most abundant group of transcription factors described in plants. Members control processes such as epidermal cell differentiation, stomatal aperture, flavonoid synthesis, cold and drought tolerance and pathogen resistance. No genome-wide characterization of this family has been conducted in a woody species such as grapevine. In addition, previous analysis of the recently released grape genome sequence suggested expansion events of several gene families involved in wine quality.

U.S. Pat. No. 7,973,216 entitled, “Compositions and Methods for Modulating Pigment Production in Plants,” is directed to [an] invention relating to polynucleotides encoding novel transcription factors and to the encoded transcription factors, which are capable of regulating anthocyanin production in plants. The invention also relates to constructs and vectors comprising the polynucleotides, and to host cells, plant cells and plants transformed with the polynucleotides, constructs and vectors. The invention also relates to methods of producing plants with altered anthocyanin production and plants by the methods.

U.S. Pat. No. 8,049,069 entitled, “Genes Involved in Plant Fibre Development,” is directed to polypeptides, and polynucleotides encoding therefore, involved in the regulation of fibre initiation and/or elongation in fibre-producing plants. In particular, the present invention provides methods of altering fibre initiation in cotton making use of transcription factors, regulatory proteins or cell cycle proteins produced at or around anthesis. The invention also relates to the use of these as markers of fibre production in plants including cotton.

U.S. Patent Application 20110072539 entitled, “Compositions and Methods for Modulating Pigment Production in Plants,” is directed to polynucleotides encoding novel transcription factors and to the encoded transcription factors, which are capable of regulating anthocyanin production in plants. The invention also relates to constructs and vectors comprising the polynucleotides, and to host cells, plant cells and plants transformed with the polynucleotides, constructs and vectors. The invention also relates to methods of producing plants with altered anthocyanin production and plants by the methods.

### SUMMARY OF THE INVENTION

The present invention discloses compositions and methods of using the same that confer quality attributes of grapes and wines, such as color and astringency, which could possess redundant, overlapping and cooperative functions.

The present invention describes and classifies 108 members of the grape R2R3 MYB gene subfamily in terms of their genomic gene structures and similarity to their putative Arabidopsis thaliana orthologues. Seven gene models were derived and analyzed in terms of gene expression and their DNA-binding domain structures. Despite low overall sequence homology in the C-terminus of all proteins, even in those with similar functions across Arabidopsis and Vitis, highly conserved motif sequences and exon lengths were found. The grape epidermal cell fate clade is expanded compared with the Arabidopsis and rice MYB subfamilies. Two anthocyanin MYBA related clusters were identified in chromosomes 2 and 14, one of which includes the previously described grape color locus. Tannin-related loci were also detected with eight candidate homologues in chromosomes 4, 9 and 11.

The present invention provides a method of regulating anthocyanin production and veraison (the transition in grape berry development from acid to sugar accumulation, production of volatiles, and antioxidant pigments) in a plant cell by providing one or more plant cells; providing a small interfering RNAs (ta-siRNAs) to the one or more plant cells; targeting a transcription factor to affect the anthocyanin biosynthesis pathway to regulate anthocyanin production and veraison in the plant cell.

The present invention provides a plant having one or more constructs to manipulate one or more plant properties including a construct that codes for a small interfering RNAs (ta-siRNAs), a Trans-Acting SiRNA Gene 4 (TAS4), MIR828 or a combination thereof that targets a transcription factor to affect one or more genes involved in a biosynthesis pathway to affect one or more plant properties.

In some embodiments, the small interfering RNAs (ta-siRNAs) are TAS4-siRNA81(−) and the transcription factor comprises a set of MYB transcription factors. In other embodiments the TAS4 may be supplied. The set of MYB transcription factors may be PAP1, PAP2, MYB113 or a combination thereof. The small interfering RNAs (ta-siRNAs) may be provided by providing a miR828 to the plant cell to trigger the cleavage of a Trans-Acting SiRNA Gene 4 (TAS4) transcript to produce a small interfering RNAs (ta-siRNAs). The method may include providing a miR828 to the plant cell, wherein the miR828 triggers the cleavage of a Trans-Acting SiRNA Gene 4 (TAS4) transcript to produce a small interfering RNAs (ta-siRNAs). The anthocyanin production and veraison may affect drought and/or salt sensitivity/tolerance. In one embodiment, a construct is provided that includes a gene coding for a small interfering RNAs (ta-siRNAs), TAS4 or both are provided and can be used to form a transgenic plant or cell. The construct may be in the form of DNA or RNA.

The present invention provides a composition to manipulate one or more berry qualities comprising: a carrier comprising a construct that codes for an interfering RNA(s) (ta-siRNAs), wherein the interfering RNAs (ta-siRNAs) targets a transcription factor to affect the anthocyanin biosynthesis pathway to affect one or more berry qualities.

The present invention provides a composition to manipulate one or more properties of a wine comprising: a carrier comprising a construct that codes for an interfering RNA(s) (ta-siRNAs), wherein the interfering RNAs (ta-siRNAs) targets a transcription factor to affect the anthocyanin biosynthesis pathway to affect one or more properties of a grape and in turn affect one or more properties of a wine.

The present invention provides a method of treating Pierce's Disease in plants by providing one or more plant cells having one or more symptom of Pierce's Disease; providing an anthocyanin effector to the one or more plant cells, wherein the anthocyanin effector affects one or more transcription factor that target one or more genes; regulating the expression of the one or more genes to ameliorate at least one of the one or more symptom of Pierce's Disease.

The present invention also provides a method of treating a gram-negative bacterium Xylella fastidiosa (XF) infection in plants by providing one or more plant cells having one or more symptom of Pierce's Disease; providing an anthocyanin effector to the one or more plant cells, wherein the anthocyanin effector affects one or more transcription factor that target one or more genes; regulating the expression of the one or more genes to ameliorate at least one of the gram-negative bacterium Xylella fastidiosa (XF) infection.

The present invention provides a method of regulating plant development and secondary metabolite biosynthesis by providing one or more plant cells; providing an anthocyanin effector to the one or more plant cells, wherein the anthocyanin effector affects one or more transcription factor; regulating the expression of one or more genes using the one or more transcription factor, wherein the one or more genes affect the plant development and/or secondary metabolite biosynthesis.

The one or more anthocyanin effectors may include a small interfering RNAs (ta-siRNAs), a Trans-Acting SiRNA Gene 4 (TAS4), MIR828 or a combination thereof. The small interfering RNAs (ta-siRNAs) include TAS4-siRNA81(−). The one or more anthocyanin effectors may include a MIR828 that triggers the cleavage of a Trans-Acting SiRNA Gene 4 (TAS4) transcript to produce a small interfering RNAs (ta-siRNAs). The one or more anthocyanin effectors may include a construct coding for a TAS4. The transcription factor may include a set of MYB transcription factors selected from PAP1, PAP2, MYB113 or a combination thereof. The one or more anthocyanin effectors may include a VvTAS4a that affects a MIR828 to trigger the cleavage of a Trans-Acting SiRNA Gene 4 (TAS4) transcript to produce a small interfering RNAs (ta-siRNAs). The method may further include the step of providing a miR828 to the plant cell, wherein the miR828 triggers the cleavage of a Trans-Acting SiRNA Gene 4 (TAS4) transcript to produce a small interfering RNAs (ta-siRNAs). The method anthocyanin production and veraison affects Pierce's Disease. The one or more plant cells may include a berry cell, a bean cell, a citrus cell, a eucalyptus cell, a Japanese cedar cell, a chicory cell, a Russian Dandelion or Rubber Root cell, and combinations thereof. The one or more plant cells may include grape cells, golden kiwi cells, apple cells, poplar cells, Gerbera cells, sunflower cells, Ipomoea cells, petunia cells, monkey flower cells, cotton cells, and cocoa cells.

The present invention provides a composition to manipulate one or more plant properties including a carrier comprising a construct that codes for a interfering RNAs (ta-siRNAs), wherein the interfering RNAs (ta-siRNAs) targets a transcription factor to affect the anthocyanin biosynthesis pathway to affect one or more plant properties. The construct may be DNA or RNA. The construct may be a double-stranded RNA is cleaved to form siRNA, a double-stranded DNA that codes for an interfering RNAs (ta-siRNAs), or a combination thereof. The construct codes for a miR828 microRNA wherein the miR828 microRNA triggers the cleavage of a Trans-Acting SiRNA Gene 4 (TAS4) transcript to produce a small interfering RNAs (ta-siRNAs). The small interfering RNAs (ta-siRNAs) may be TAS4-siRNA81(−). The transcription factor may be a set of MYB transcription factors that may include PAP1, PAP2, MYB 113 or a combination thereof.

The present invention provides a plant cell having one or more constructs to manipulate one or more plant properties comprising: a construct that codes for a small interfering RNAs (ta-siRNAs), a Trans-Acting SiRNA Gene 4 (TAS4), MIR828 or a combination thereof that targets a transcription factor to affect one or more genes involved in a biosynthesis pathway to affect one or more plant properties.

The present invention provides a method of treating one or more symptom associated with Pierce's Disease in plants by providing one or more plant cells having one or more symptom of Pierce's Disease; providing an anthocyanin effector to the one or more plant cells, wherein the anthocyanin effector affects one or more transcription factor that target one or more genes; and regulating the expression of the one or more genes to ameliorate at least one of the one or more symptom of Pierce's Disease.

### BRIEF DESCRIPTION OF THE DRAWINGS

For a more complete understanding of the features and advantages of the present invention, reference is now made to the detailed description of the invention along with the accompanying figures and in which:

FIGS. 1a-1d are images of the sequence from Arabidopsis thaliana of the promoters of TAS4 (a), MIR828 (b), MYB75 (c) and primer sequences used to study the subject (d).

FIG. 2a is a schematic of PAP1 and PAP2 genes with qRT-PCR primer pairs mapped.

FIG. 2b is a table of the time course of sucrose, Glucose, or mannitol treatments of Col-0 seedling at a concentration of 100 mM up to 24 hours.

FIG. 2c and FIG. 2d are tables showing the sucrose response of ABA mutant genotypes treated for 24 hours.

FIGS. 3a and 3b are images of the physiological concentrations of sucrose and Glucose that induce expression of TAS4-siR81(−).

FIGS. 4a and 4b are images of a negative feedback regulatory loop involving PAP1 and TAS4-siR81(−).

FIG. 5 is a graph of the sucrose treatment that induces anthocyanin accumulation in ta-siRNA pathway mutants.

FIG. 6 is an image of a sequence alignment of TAS4 paralogs in dicot plants.

FIG. 7 is an image of an amino acid sequence alignment of miR828 complementary sites in PAP1 orthologs from diverse flowering plant genera.

FIG. 8 is an image of an amino acid sequence alignment of TAS4-siR81(−) complementary sites in PAP1 orthologs from diverse flowering plant genera.

FIG. 9 is an image of the gymnosperm P. glauca predicted pri-miR828 transcript.

FIGS. 10a-10b are images of the two predicted alternative secondary structures with similar delta-G free energies form “good” hairpin structure which could generate mature miR828 from either of these candidate loci.

FIG. 11a is an image of a sequence alignment for MIR828 genes from dicot, monocot and gymnosperm species.

FIG. 11b is an image of an extended sequence alignment of candidate P. contorta MIR828 gene and three predicted MYB targets from P. contorta.

FIG. 11c is an image of a sequence alignment for MIR828 gene and TAS4 in A. thaliana showing homologies suggestive of a common evolutionary lineage.

FIG. 11d is an image of 5′-modified RACE clones establishing cleavage of a P. resinosa MYB target mRNA by miR828.

FIG. 12 is an image of the feedback regulatory loop involving PAP1/MYB75 and TAS4 in response to sugars in Arabidopsis.

FIG. 13 is an image of the sequence of the miR828 and to-siR81(−) footprints showing selective sweep in grapes.

FIG. 14 is a graph of the expression of TAS4 genes in grape deduced from sRNA-seq read abundance showing TAS4ab are highly expressed in grape leaf and flower but not fruit and that miR828b is expressed in leaf.

FIGS. 15a-15e are images of the sequence that predicts targets of Vv-TAS4-siR81 (−).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term TAS denotes a trans-acting siRNA gene.

As used herein, the term miRNA denotes a microRNA.

As used herein, the term Suc denotes Sucrose.

As used herein, the term Glc denotes Glucose.

As used herein, the term PAP1 denotes Production of Anthocyanin Pigment 1.

As used herein, the term ABA denotes abscisic acid.

As used herein, the term sRNA denotes a Small RNA.

The present invention discloses a model plant Arabidopsis a sugar-, phosphate-, and abscisic acid (ABA) auto-regulatory loop involving microRNA828 (miR828) and Trans-Acting-Small RNA locus4 (TAS4). In the overlapping plant RNA interference pathways that include miRNAs and TAS genes, 21-nucleotide duplexes of siRNAs are processed from longer double stranded RNA precursors. Single-stranded siRNAs then guide an RNA-induced silencing complex to execute posttranscriptional silencing of complementary target messenger RNAs. We have shown that MIR828 and TAS4 are structurally and functionally related non-coding RNA loci which down-regulate anthocyanin biosynthesis by Watson-Crick base pairing to, and subsequent endonucleolytic cleavage of, transcripts encoding novel MYB class transcription factors.

The target MYB genes in grape are key effectors of anthocyanin accumulation in Pierce's Disease (PD) etiology and véraison (the transition to ripening), mediated through Pi sensing crosstalk that modulates miR828 and TAS4 activities normally to silence target MYB expression. There is expression evidence that VvTAS4 dramatically decreases during grape berry development, supporting the hypothesis of miR828/TAS4 regulation may be the cause of anthocyanin accumulation in Xylella infection, phosphate effects on berry development, and production of color and flavor enhancers and antioxidant flavanoids in berries.

The present invention also provides methods and compositions using digital expression (NanoString) technology to quantify miR828, TAS4 siRNAs and cognate target transcription factor transcripts and marker genes induced by infections, phosphate starvation, and ripening.

Methods have been described in publications by the PI and Co-Is. NanoString nCounter multiplex technology incorporates fluorescent barcodes together with a digital readout for single-molecule imaging. It does not involve reverse transcription or any other enzymatic reaction; instead the technology relies on sequence-specific probes to digitally measure target or small RNA abundance (i.e., mRNA or miRNA) within a given sample including plants. Each custom CodeSet will be designed and synthesized at NanoString Technologies. Total RNA (100 ng) from plant material will be analyzed using the NanoString nCounter Analysis System. All procedures related to mRNA and small RNA quantification including sample preparation, hybridization, detection, and scanning will be carried out as recommended by the manufacturer.

miR828 in Arabidopsis triggers the cleavage of Trans-Acting SiRNA Gene 4 (TAS4) transcripts and production of small interfering RNAs (ta-siRNAs). One siRNA, TAS4-siRNA81(−), targets a set of MYB transcription factors including PAP1, PAP2, and MYB 113 which regulate the anthocyanin biosynthesis pathway. miR828 also targets MYB113, suggesting a close relationship between these MYBs, miR828, and TAS4. PAP1, PAP2, and TAS4 expression is induced specifically by exogenous treatment with sucrose and glucose in seedlings. The induction is attenuated in abscisic acid (ABA) pathway mutants, especially in abi3-1 and abi5-1 for PAP1 or PAP2, while no such effect is observed for TAS4. PAP1 is under regulation by TAS4, demonstrated by the accumulation of PAP1 transcripts and anthocyanin in to-siRNA biogenesis pathway mutants. TAS4-siR81(−) expression is induced by physiological concentrations of Suc and Glu and in pap1-D, an activation-tagged line, indicating a feedback regulatory loop exists between PAP1 and TAS4. Bioinformatic analysis revealed MIR828 homologues in dicots and gymnosperms, but only in one basal monocot, whereas TAS4 is only found in dicots. PAP1, PAP2, and MYB113 dicot paralogs show peptide and nucleotide footprints for the TAS4-siR81(−) binding site, providing evidence for purifying selection in contrast to monocots. Extended sequence similarities between MIR828, MYBs, and TAS4 support an inverted duplication model for the evolution of MIR828 from an ancestral gymnosperm MYB gene and subsequent formation of TAS4 by duplication of the miR828 arm. This is illustrated by modified 5′-RACE for a MYB mRNA cleavage product guided by miR828 in Pinus resinosa. Taken together, the regulation of anthocyanin biosynthesis by TAS4 and miR828 in higher plants is evolutionarily significant and consistent with the evolution of TAS4 since the dicot-monocot divergence.

Trans-Acting siRNA (TAS) genes are small interfering RNA (siRNA)-generating loci that regulate target gene expression in trans. The production of trans-acting siRNAs (ta-siRNAs) from TAS loci depends on microRNA (miRNA)-directed cleavage of their transcripts by argonaute (AGO)-containing RNA-Induced Silencing Complexes (RISCs), which sets the phase for 21-nt siRNA production by RNA-dependent RNA polymerase 6 (RDR6) in collaboration with suppressor of gene silencing 3 (SGS3), dicer-like 4 (DCL4), double-stranded RNA binding protein 4 (DRB4), and HUA enhancer 1 (HEN1), a small RNA (sRNA) methyltransferase. Arabidopsis thaliana has eight TAS loci from four families, TAS1-4. TAS1 and TAS2 transcripts are subject to miR173-directed cleavage in association with AGO1 to generate siRNAs targeting several transcripts of pentatricopeptide repeat-containing genes and others with unknown function. TAS3 transcript, on the other hand, is cleaved through the specific interaction of miR390 with AGO7. An autoregulatory network has been found involving miR390, TAS3, and to-siRNA targets auxin response factors 2 (ARF2), ARF3, and ARF4. TAS3-derived siRNAs (ta-siARFs) inhibit ARF2/3/4 expression, while ARF4 down regulates miR390 accumulation in contrast to the upregulation of miR390 by ARF3 in response to auxin. The outcome of this complex feedback loop is a fine-tuning of lateral root growth dependent on the auxin receptor transport inhibitor response 1 (TIR1; a target of miR393), which directs transcriptional regulation in response to localized auxin fluxes. Regulatory networks of sRNAs, including miRNAs and ta-siRNAs, modulate their targets' expression in response to primary (N, P, K) and secondary (S, Mg, Ca) macronutrient condition changes in the cell and/or environment. For example, low sulfate induces miR395 expression, which decreases the mRNA level for its targets ATP sulfurylase 1 (APS1) and several other genes in the sulfate assimilation pathway. The induction of miR395 is modulated by sulfur limitation 1 (SLIM1), a putative transcription factor in the same pathway, although the expression domain for such induction does not correlate with one of its targets, sulfate transporter 2;1 (SULTR2;1)/Arabidopsis Sulfate Transporter 68 (AST68). Other examples include phosphate (Pi) starvation, which up-regulates miR399b/c/f expression and down-regulates their common target phosphate 2 (PHO2)/ubiquitinconjugating enzyme 24 (UBC24). Transgenic Arabidopsis plants overexpressing MIR399 accumulate five to six times more Pi in shoots than wild type. Intriguingly, a non-coding RNA, induced by phosphate starvation 1 (IPS1) can sequester miR399 by base-pairing through a mechanism termed “target mimicry” and thereby up-regulate PHO2 expression level to help translocate over-accumulated Pi in shoots. Deep sequencing techniques have uncovered miRNAs such as miR398, miR778, miR827, and miR2111 responsive to Pi deficiency. Several members of the miR169 family and miR398a are repressed by nitrogen (N) limitation (Pant et al. 2009).

Another nutrient-responsive example for miRNAs comes from the report that exogenous sucrose (Suc) treatment increases levels of miR398 in a dose-dependent, but not time-dependent manner, probably by activating the transcription of miR398c (Dugas and Bartel 2008). miR398 reduces the expression of its targets, including copper superoxide dismutase 1 (CSD1) and CSD2 at both mRNA and protein levels. Production of anthocyanin pigment 1 (PAP1)/MYB domain protein 75 (MYB75) and PAP2/MYB90 encode transcription factors that regulate expression of anthocyanin biosynthetic genes in vegetative tissues. They might be involved in regulating leaf senescence because for both of these processes, sugars can be triggers. In this study, we report that TAS4 and its targets PAP1 and PAP2 are responsive to Sucrose. Part of the response is impaired in ABA insensitive 3 (abi3) and abi5 mutants. PAP1 and TAS4 expression appear to involve in an autoregulatory loop, as evidenced by the over-accumulation of PAP1 transcript levels and anthocyanin in ta-siRNA pathway mutants, and the up-regulation of TAS4-siR81(−) in pap1-D, an activation-tagged transgenic line. We also performed bioinformatic analysis and uncovered the existence of miR828 in gymnosperms and angiosperms, whereas TAS4 only is found in dicots. The cleavage by miR828 was mapped on one MYB transcript from Pinus resinosa by modified 5′-RACE.

Finally, sequence alignments suggest an inverted duplication model for miR828 and TAS4 evolution. Materials and methods Plant materials and growth conditions Arabidopsis (Arabidopsis thaliana ecotype Columbia) wild-type and mutant plants were grown as previously described (Luo et al. 2009). The accessions used in this study are listed as follows: Ler-0 [CS20], abi1-1 [CS22], abi2-1 [CS23], abi3-1 [CS24], abi5-1 [CS8105], aba1-1 [CS21], Col-0 [CS60,000], abi4-103 [CS3838], hen1-1 [CS6583], dcl4-2 [CS6954], drb4 [SALK_{—}113384c], rdr6-15, sgs3-14 [SALK_{—}001394], tas4 [SALK_{—}066997], mir828 [SALK_{—}097788], hyl1-2 [SALK_{—}064863], hst-6 [CS24279], hst-7 [CS24280], and pap1-D [CS3884] (Borevitz et al. 2000; Alonso et al. 2003). For the treatment with sugars, 3-day-old Arabidopsis seedlings were grown on filter papers supplemented with Murashige and Skoog standard medium (MS medium, one half strength hours, control). Half of the samples were transferred to new filter papers supplemented with Sucrose, glucose (Glc) or mannitol solutions at a concentration of 100 mM and harvested by freezing in liquid N2 at various time points up to 24 hours, or subjected to the treatment of different sugars for 12 hours with a series of concentrations ranging from 0 to 100 mM. Taxus globosa (Mexican yew) and Pinus resinosa (red pine) plants were purchased from Forrest Farm (Williams, Oreg.) and Heronswood Nursery (Warminster, Pa.), respectively, and total RNA was extracted from green needles as described (Chang et al. 1993) for Rapid Amplification of cDNA Ends (RACE) studies. RNA preparation and detection Total RNA was isolated using Trizol regent (Invitrogen, Carlsbad Calif.). Northern blots and sRNA blots were performed as described (Xie et al. 2005). High molecular weight RNA was precipitated from total RNA with 2 M LiCl followed by centrifugation (13,000 rpm, 15 min). The supernatant was added to three volumes 100% ethanol to precipitate low molecular weight RNA. Ten μg total RNA or 20 μg low molecular weight RNA was loaded in each lane for formaldehyde-agarose or PAGE gel electrophoresis, respectively. For Northern blots, probes were prepared from agarose gel-purified PAP1 cDNA from Arabidopsis cDNA library amplified using primers “PAP1_atgF” and “PAP1_tagR” and radio-labeled with α-32P-dCTP by a random primer labeling kit (Takara, Shiga Japan). To check equal loading, the membrane blot was stripped and re-probed with antisense γ-32P-labelled oligonucleotides for miR160, 5S rRNA, and/or U6 small nuclear RNA (snRNA). RNA blots were scanned using a Storm 860 PhosphorImager (GE Healthcare, Piscataway N.J.). mRNA or sRNA signals were quantified.

Specifically, we divided the TAS4-siR81(−), PAP1, and control (5S rRNA, miR160, or U6 snRNA) band areas into nine vertical subsections of equal area per lane. The paired subsections for signals of a given lane were integrated separately after subtracting representative background fields flanking the test and control bands. A ratio of TAS4-siR81(−) or PAP1 mRNA signals to various controls was calculated for these independent sections. The average of four to six uniform ratios across the band was calculated after discarding subsections that contained artifacts identified visually and attributed to gel or blotting processes. Modified RACE studies were performed according to the manufacturer's specification. Cloned cDNAs encoding MYB homologues obtained from RACE studies on P. resinosa and T. globosa were submitted to GenBank (accession numbers HQ997774 and HQ997775, respectively).

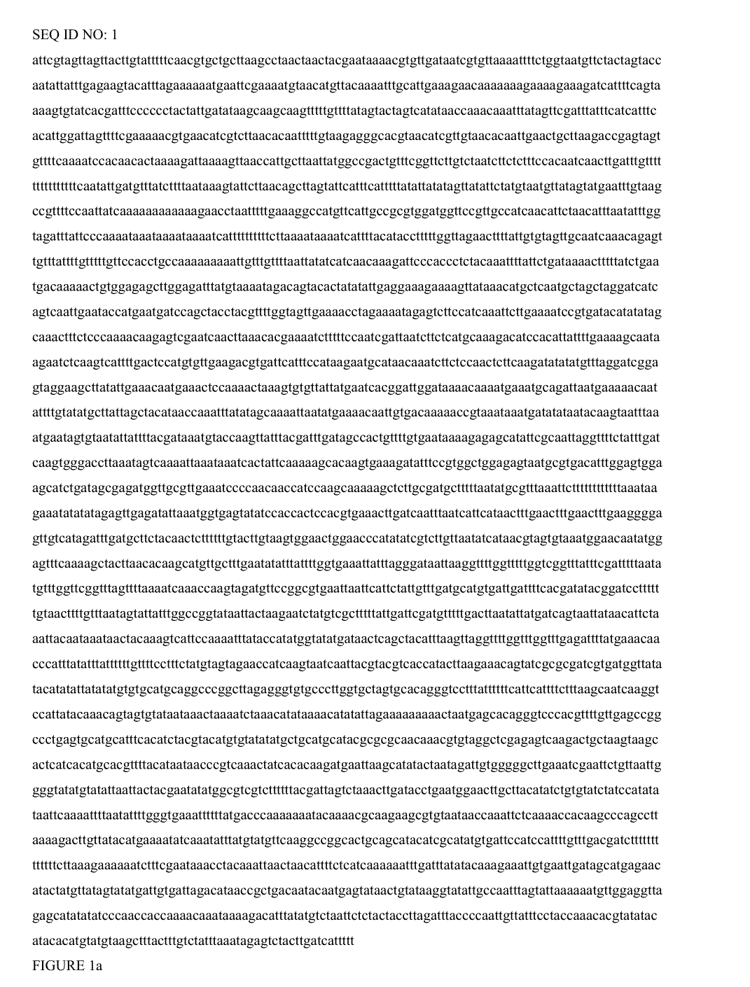

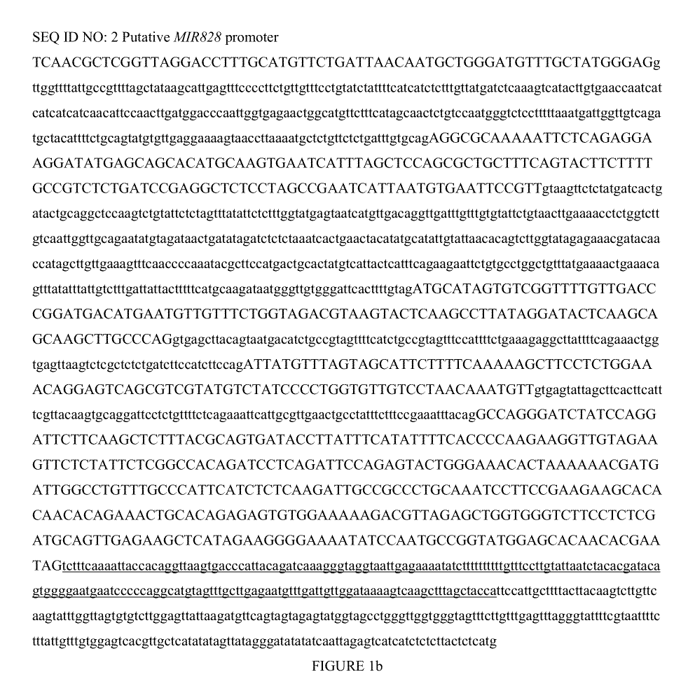

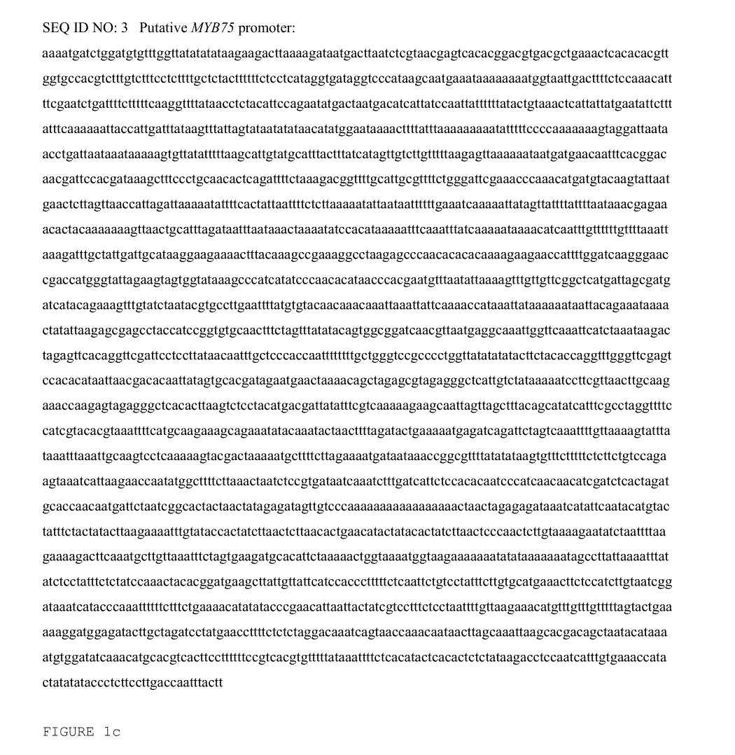

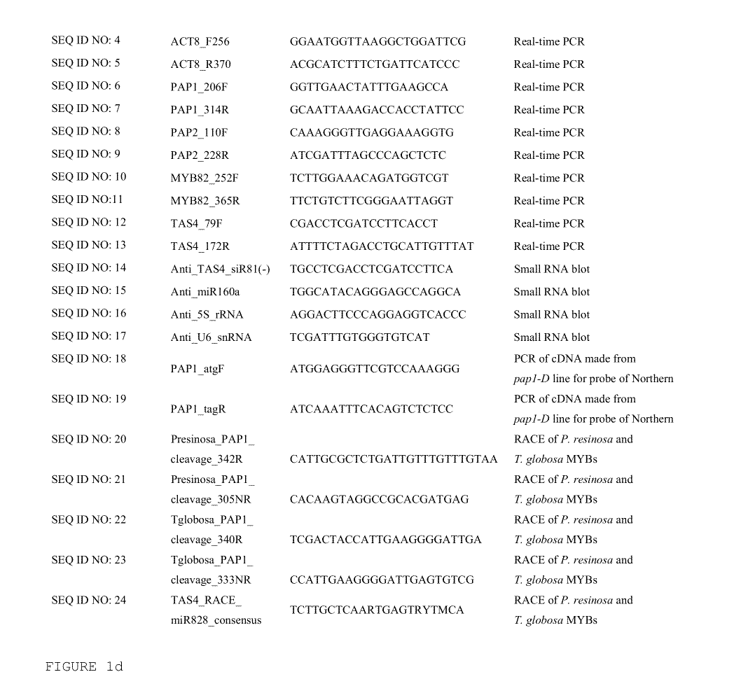

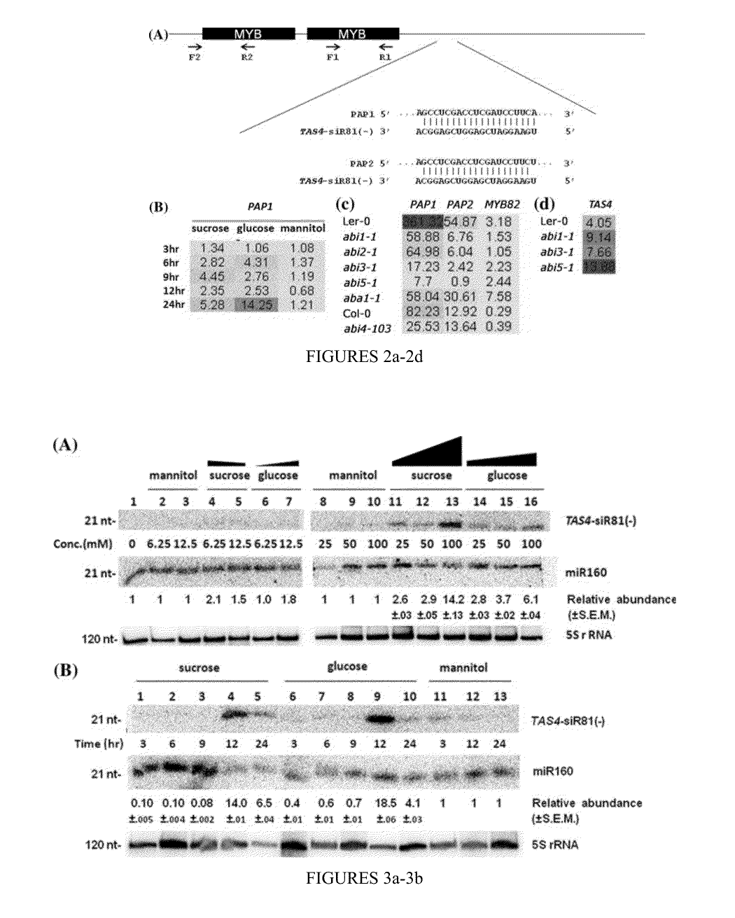

FIGS. 1a-1d are images of the gene promoter and probe primer sequences. SEQ ID NO: 1 is a listing of the gene promoter. FIG. 1b is the Putative MIR828 promoter SEQ ID NO: 2. FIG. 1c is the Putative MYB75 promoter SEQ ID NO: 3. FIG. 2d is a table of the sequences, SEQ ID NO: 4-24. Real-time RT-PCR RNA was extracted from seedlings grown on 0.59 MS medium (control) or on the same medium with 100 mM sugars added for a series of time points as described in Figure legends. Total RNA was subjected to DNase I treatment (Promega, Madison Wis.) after extraction by Trizol solution (Invitrogen). Five micrograms of each sample were reverse-transcribed into cDNA with Oligo dT primers (Promega) by Moloney Murine Leukemia Virus Reverse Transcriptase (Promega) for 1 hours at 42° C. Quantitative realtime PCR (qRT-PCR) assay was performed. ACTIN8 primer pairs were used for internal control on aliquots of cDNA. Relative quantitation for gene expression was done using the comparative CT method as described in the ABI Prism 7300 Sequence Detection System User Bulletin (Applied Biosystems). Anthocyanin quantitation Extraction and quantification of anthocyanin from Arabidopsis seedling was performed as described with minor modifications. In brief, 10-20 three day-old seedlings were placed in a microcentrifuge tube and centrifuged briefly to allow surface liquid to be pipetted off. The samples were weighed twice on an analytical balance to obtain an average fresh weight of tissue. One mL of extraction buffer (1% [v/v] hydrochloric acid in methanol) was added followed by incubation at 4° C. for 24 hours. Extracts were centrifuged (15 min at 13,000 rpm) and the absorbance of the supernatant was determined at 530 and 657 nm in a BioMate 5 spectrophotometer. Relative anthocyanin units are defined as equal to one absorbance unit [A530-(1/4 9 A657)] 9 1,000] per gram fresh material in one mL of extraction buffer. Mean values were obtained from three biological replicates. Bioinformatic analysis Expressed Sequence Tags (ESTs) and protein sequences were obtained by BLASTing from GenBank. The alignment was performed with the Vector NTI software package or T-Coffee.

FIGS. 2a-2d qRT-PCR shows temporal induction of PAP1, PAP2 and TAS4 by Suc and Glu, and crosstalk with ABA signaling. FIG. 2a is schematic of PAP1 and PAP2 genes with qRT-PCR primer pairs mapped (F1, R1 for PAP1; F2, R2 for PAP2, respectively). The base pairing between TAS4-siR81(−) (SEQ ID NO: 26) with PAP1 (SEQ ID NO: 25) or PAP2 (SEQ ID NO: 27) is shown underneath hours.

FIG. 2b is table of the time course of Suc, Glu, or mannitol treatments of Col-0 seedling at a concentration of 100 mM up to 24 hours. Each treatment is represented by a column of colored boxes, and each time point is indicated by an individual row. For Sucrose treatment of Arabidopsis seedlings, 3-day-old seedlings were grown on filter papers supplemented with Murashige and Skoog (MS) standard medium (½ strength hours, control). Data (average transcript level from three technical replicates) were visualized using BAR HeatMapper Plus software. Data are represented as fold change (unity=control) after normalization to ACTIN8 expression. Effects of different sugars on gene expression range from pale yellow (low) to deep red (high). The study was performed twice with similar results.

FIG. 2c and FIG. 2d are tables showing the 100 mM Sucrose response of ABA mutant genotypes treated for 24 hours. The expression data for each gene is represented by a column of colored boxes, while each genotype assayed is indicated by an individual row.

Secondary structures of RNAs were predicted using MFOLD (Zuker 2003). Sugar induction of PAP1 and TAS4 expression PAP1 and PAP2 are predicted targets of TAS4-siR81(−) (FIG. 2a). Using qRT-PCR, PAP1 expression was assayed in response to sugars. In a timecourse treatment with exogenous sugars of Col-0 seedlings, PAP1 expression was induced by Sucrose and Glucose up to *5- and 14-fold, respectively, whereas PAP1 was not induced by the non-metabolizable sugar mannitol used as a control (FIG. 2b). PAP2 showed a similar but lower induction than PAP1 by Sucrose treatment (*6-fold less than PAP1; see FIG. 2c, the rows for “Ler-0” and “Col-0”). Abscisic acid (ABA) signaling synergizes with sugar and induces anthocyanin accumulation in early seedling development (Rolland et al. 2006; Finkelstein et al. 2002). Several sugar-insensitive mutants were isolated as allelic to ABA synthesis (aba) and ABA insensitive (abi) mutants. For example, sucrose insensitive 10 (sis10) was cloned in a forward genetic screen and shown to be allelic to ABI3, encoding a B3 domain transcription factor that confers sugar and ABA sensitivity and regulates anthocyanin production (Parcy et al. 1997; Huang et al. 2008). Sucrose uncoupled 6 (sun6), sugar-insensitive 5 (sis5), glucose insensitive 6 (gin6), and impaired Sucrose induction 3 (isi3) are mutant alleles of ABI4, which encodes an APETALA2 domain transcription factor (Huijser et al. 2000; Laby et al. 2000; Arenas-Huertero et al. 2000; Rook et al. 2001). To measure the effects of sugar induction on PAP1 and TAS4, qRT-PCR assays were performed on samples from mutants in ABA biosynthesis and signaling pathways (FIGS. 2c, 2d). Interestingly, in abi1-1, abi2-1, abi4-103, and aba1-1 mutants the induction of PAP1 and PAP2 by Sucrose was significantly reduced (*2- to 8-fold) compared to wild type Ler-0 or Col-0, although still effectively Suc-responsive (FIG. 2c). This indicated the positive effect of ABA signaling and biosynthesis on PAP1/PAP2 responses to Suc. In abi3-1 and abi5-1 mutants, PAP1 expression upon Sucrose treatment was severely decreased compared to wild type (*21- and 47-fold less, respectively). In addition, PAP2 barely responded to Sucrose treatment in abi3-1 and abi5-1 mutants. These results are generally consistent with the sugar-insensitive phenotypes associated with abi3, abi4 and abi5 mutants. Interestingly, the expression of MYB82, a PAP1 paralog which has a predicted but un-validated miR828 complementary site, did not respond to Sucrose in wild type or mutants. However, TAS4 expression was increased two to threefold by Sucrose in the abi1-1, abi3-1 and abi5-1 mutants in comparison to Ler-0, suggesting its expression is independent of the ABA signaling pathway or subject to secondary effects (FIG. 2d).

FIGS. 3a and 3b are images of the physiological concentrations of Suc and Glu induce expression of TAS4-siR81(−). FIG. 3a is an image of a sRNA blot of RNA from 3-day-old Col-0 wild-type seedlings were grown on filter papers supplemented with MS medium (½ strength hours, control) and then subjected to treatment with different sugars in series of concentrations ranging from 0 to 100 mM for 12 hours. FIG. 3b is an image of the timecourse study from 3 to 24 hours treatments with 100 mM Sucrose, Glucose or mannitol. As loading controls, probes for 5S rRNA and miR160 were hybridized to the same membrane. Band intensities for TAS4-siR81(−) are shown normalized to that of miR160 below each lane (±standard error of mean) and graphically as ‘effect wedges’ above the treatment headers. The relative abundances for TAS4-siR81(−) are presented as the ratio of normalized abundance from Sucrose or Glucose treatments to that from respective mannitol controls (set to unity). A representative result from three studies is shown. sRNA blots showed that TAS4-siR81(−) was induced strongly in a dose-dependent manner by exogenous Sucrose treatment for 12 hours (FIG. 3a). The expression of TAS4-siR81(−) was induced by physiological concentrations of 6.25 mM Sucrose or 12.5 mM Glucose (Tang and Sheen 1994) relative to a corresponding control (2.1-, and 1.8-fold higher than mannitol control, respectively). Clear signals corresponding to TAS4-siR81(−) were detected for samples treated with 25 mM Sucrose (2.6-fold higher than that in samples treated by mannitol), with maximum signal intensities observed for samples treated with 100 mM Sucrose for 12 hours (14-fold higher than mannitol control). Increasing Glucose concentrations had similar effects as Sucrose on TAS4-siR81(−) expression (*3- to 6-fold induction after 12 h), while the non-metabolized osmolyte mannitol (a negative control) had a very weak effect, indicating that TAS4-siRNA81(−) induction is primarily due to metabolizable sugars and that the mannitol effect observed at high concentrations may be an osmotic stress-related response (FIG. 3a, data not shown).

As the basis for quantifying endogenous sRNA abundance, miR160 and 5S rRNA expression were shown to be independent of sugar treatments, which supports the specificity of Sucrose and Glucose induction for TAS4-siR81(−) expression (FIG. 3a, 3b). The response of TAS4-siR81(−) to Sucrose or Glucose was also transient, reaching a peak at 12 hours (*14- and 18-fold induction by Sucrose and Glucose, respectively) with subsequent declines in abundance at 24 hours (*6- and 4-fold induction by Sucrose or Glucose, respectively, FIG. 3b), suggesting a homeostatic mechanism involving the expression of TAS4. An autoregulatory feedback loop involving PAP1 and TAS4 regulates anthocyanin production PAP1 is predicted to carry a functional TAS4-siR81(−) target site. Its regulation by RISC is supported by qRT-PCR studies showing up-regulation in mir828 and tas4 T-DNA insertion mutants.

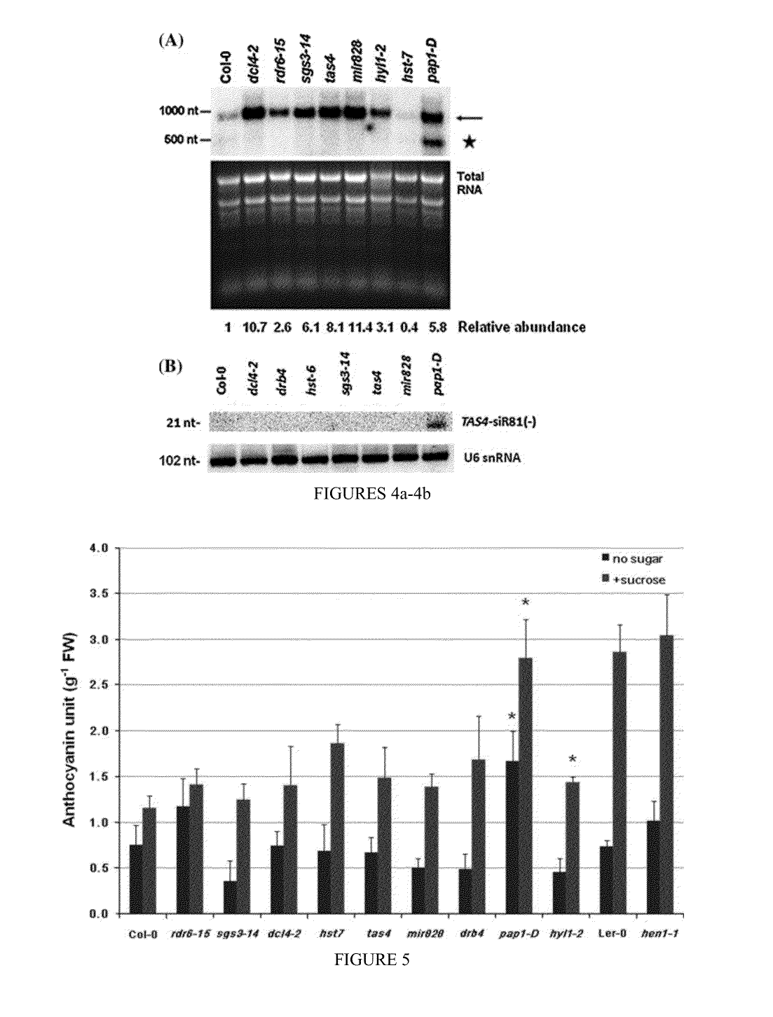

FIGS. 4a and 4b are images of a negative feedback regulatory loop involving with PAP1 and TAS4-siR81(−). FIG. 4a is an image of an RNA blot from 3-day-old Col-0 wild-type seedlings were grown on filter papers supplemented with ½ strength MS medium (control) and then subjected to treatment with 100 mM sucrose for 12 hours. The arrow indicates a band corresponding to the full length mRNA for PAP1, and the star shows a signal with the correct predicted size of the TAS4-siR81(−)-mediated 3′ cleavage product of PAP1. Total RNA (10 μg) was loaded for each sample and stained with ethidium bromide before blotting to confirm equal loadings. The relative abundance for PAP1 is presented below the gel as the ratio of band intensities for each mutant versus that from wild type Col-0.

FIG. 4b sRNA blot analysis for TAS4-siR81(−) expression in tasiRNA pathway mutants, a mir828 T-DNA insertion mutant, and a pap1-D over-expressing activation-tagged transgenic line. Low molecular weight RNA (20 μg) was loaded for each lane. The same membrane was re-hybridized with a probe against U6 snRNA to show equal loading. FIG. 4a (arrow) shows that PAP1 mRNA was elevated from 2.6- to 10.7-fold in these mutant seedlings in response to treatment with sucrose for 12 hours, as well as in miR828 and tas4 mutants (11.4- and 8.1-fold increases, respectively). In pap1-D, a dominant activation tagged transgenic line, PAP1 expression was elevated compared to wild type (5.8-fold induction). Interestingly, there was a band of size ˜450 nt (asterisk in FIG. 4a) presumed to be the TAS4-siR81(−)-directed 3′ cleavage product of PAP1 mRNA, based on similar phenomena observed for many miRNA targets (Souret et al. 2004). The cleavage product was just barely visible in Col-0, dcl4-2, and hst-7.

The accumulation of both PAP1 mRNAs and its 30′ cleavage product in pap1-D suggests that increased PAP1 mRNA levels may enhance post-transcriptional regulation of itself by TAS4-siR81(−). A sRNA blot confirmed that TAS4-siR81(−) expression was below detection levels in wild type and all ta-siRNA pathway mutants assayed, but significantly increased in pap1-D (FIG. 4b). Taken together, these results suggest that an autoregulatory feedback loop involving PAP1 and TAS4-siR81(−) operates on and coordinates TAS4 expression. Supporting this notion, two putative PAP1-binding motifs (C/T)(A/C)NCCACNN(G/T) (SEQ ID NO: 89) were found within the 2,000 nt region upstream of the TAS4 transcription start site, according to PAP1 cis regulatory elements functionally characterized by transient assays in protoplasts as seen in FIG. 1a-c. dcl4-2 and drb4-1 mutants over-accumulate anthocyanin in leaves and flowers of plants older than 6 weeks.

FIG. 5 is a graph showing that the sucrose treatment induces anthocyanin accumulation in ta-siRNA pathway mutants. Three-day-old Arabidopsis seedlings were grown on filter papers supplemented with MS medium (½ strength), half of which were transferred to new filter papers supplemented with 100 mM Sucrose for 12 hours and the rest treated with H₂O. Data from one of two representative studies is shown. Error bars are standard errors of mean (n=3 biological replicates). Asterisks indicate significantly higher anthocyanin than wild type control (P=0.06, one-sided Student's t-test, equal variance assumed). To find out the effect of Sucrose treatments, we assayed the accumulation of anthocyanin in various tasiRNA pathway mutants (FIG. 5). With the exception of pap1-D, untreated 3 day-old mutant seedlings did not accumulate significantly different amounts of anthocyanins than their corresponding wild types (FIG. 5, blue bars). After 12 hours, Sucrose treatment, all mutants displayed increased accumulation of anthocyanin compared to their non-treated seedlings (FIG. 5, red bars), consistent with previous findings. Like untreated pap1-D mutant results, pap1-D seedlings had the highest anthocyanin accumulation after treatment, with hyl1-2 mutants also accumulating significantly higher amounts of anthocyanins compared to wild type Col-0 (FIG. 5, asterisks). All other tested ta-siRNA pathway mutants accumulated higher amounts of anthocyanin than wild types. These results suggest that the release of PAP1 repression by loss of TAS4-siR81(−) (FIG. 4ab) in the mutants could be responsible for increased anthocyanin under Sucrose stimulus conditions (FIG. 5).

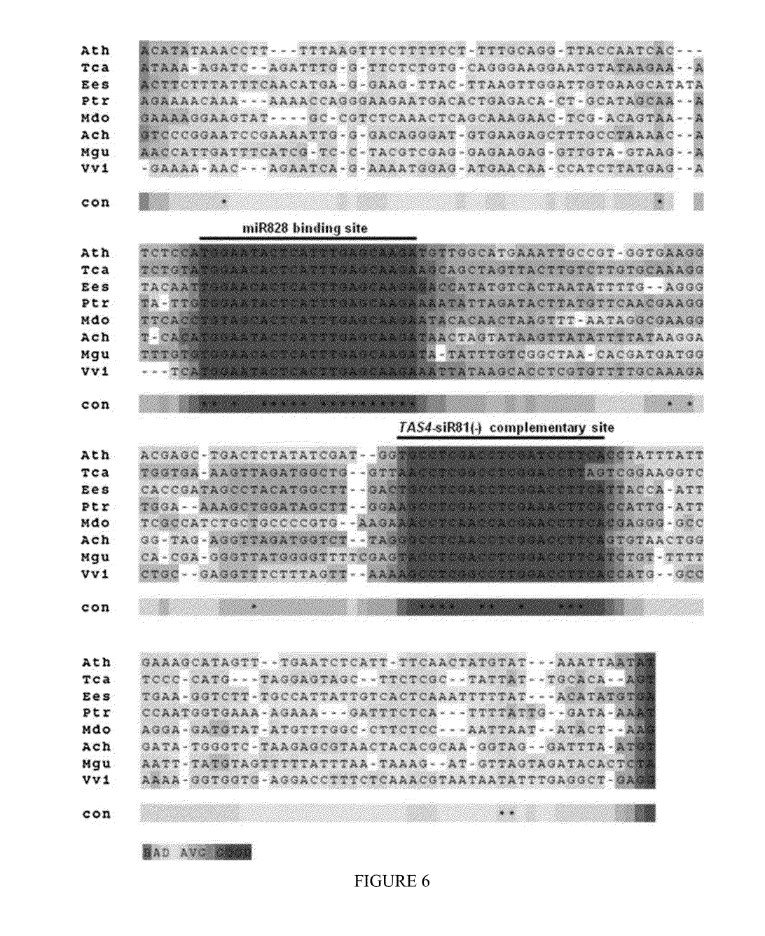

FIG. 6 is an image of a sequence alignment of TAS4 paralogs in dicot plants. Sequences were obtained by BLASTing TAS4/AT3G25795 (n.t. 870-980) against the GenBank plant EST database. Alignments were color-coded based on the confidence of the local alignment of T-Coffee (yellow\brown\red). The putative miR828 binding site and TAS4-siR81(−)-generating site are labeled with black lines. Asterisks show residues identical for the given position. Abbreviations correspond to species are listed as follows with TAS4 paralog (GenBank accession numbers). SEQ ID NO: 28 At, Arabidopsis thaliana; SEQ ID NO: 29 Tca, Theobroma cacao (CU512683.1); SEQ ID NO: 30 Ees, Euphorbia esula (DV114602.1); 31 Ptr, Populus tremula (DN495932.1); SEQ ID NO: 32 Mdo, Malus domestica (CN490819.1); SEQ ID NO: 33 Ac, Actinidia chinensis (FG511890.1); SEQ ID NO: 34 Mgu, Mimulus guttatus (DV209191.1), and SEQ ID NO: 35 Vvi, Vitis vinifera (EC986896.1). Con consensus, the same nucleotide on one position is represented by asterisk.

Evolution of TAS4 and its regulator miR828 Bioinformatic analysis of ESTs in land plants demonstrated the existence of TAS4 in dicots, such as Euphorbia esula, Actinidia chinensis, and Vitis vinifera (FIG. 6). The TAS4 orthologs bear conserved miR828 binding sites, whereas a less-conserved TAS4-siR81(−) complementary site is located downstream by a constant distance of four 21-nt phases (FIG. 6 black lines), despite the sequence divergence in the intervening region. These data clearly show that a “selective sweep” has acted over evolutionary time on miR828 and TAS4-siR81(−) sequences to maintain the function of TAS4 in these species.

FIG. 7 Amino acid sequence alignment of miR828 complementary sites in PAP1 orthologs from diverse flowering plant genera (SEQ ID NOS: 36-50). Sequences were obtained by BLASTing the Arabidopsis PAP1 sequence to the GenBank protein database. The alignment was done by Vector NTI (Invitrogen, version 9). A cartoon for MYB ortholog conserved domain structure is shown above the alignment. The miR828 complimentary sites are labeled by a bracket and the conserved residues are shaded.

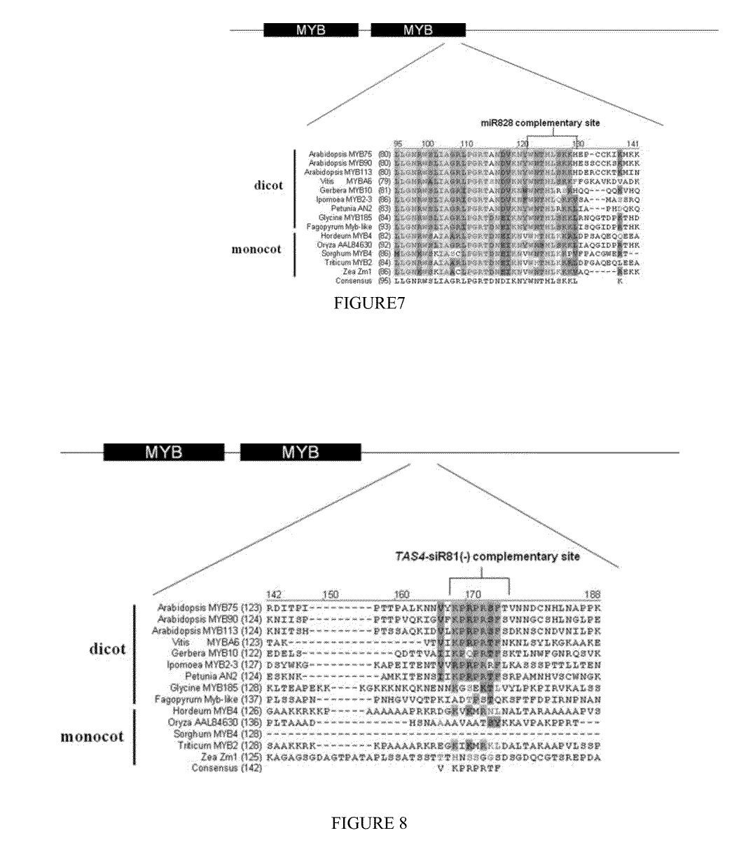

Supporting evidence was found by alignment of sequences for PAP1/PAP2/MYB113 orthologs which show the peptide footprint for miR828 binding sites is generally conserved for both dicot and monocot plants (FIG. 7), while that for TAS4-siR81(−) binding sites is specific for most dicots only (as seen in FIG. 8).

FIG. 8 Amino acid sequence alignment of TAS4-siR81(−) complementary sites in PAP1 orthologs from diverse flowering plant genera (SEQ ID NOS: 51-65). DNA sequence alignment revealed a MYB-like gene in Fagopyrum as potential target for miR828, based on sequence similarity with miR828 complementary site in Arabidopsis MYB113. MYBA6 in Vitis is also predicted as TAS4 target. These observations support purifying selection for miR828 and TAS4 regulation on individual MYB targets in different dicot species as shown initially in Arabidopsis. By searching plant EST databases, MIR828 orthologs with extensive base pairing to form hairpins were found in a variety of dicot species, including A. lyrata, E. esula, V. vinifera, Gossypium raimondii (data not shown) and gymnosperms Picea glauca (spruce) and Pinus contorta (lodgepole pine) (FIG. 8a and FIGS. 9 and 10).

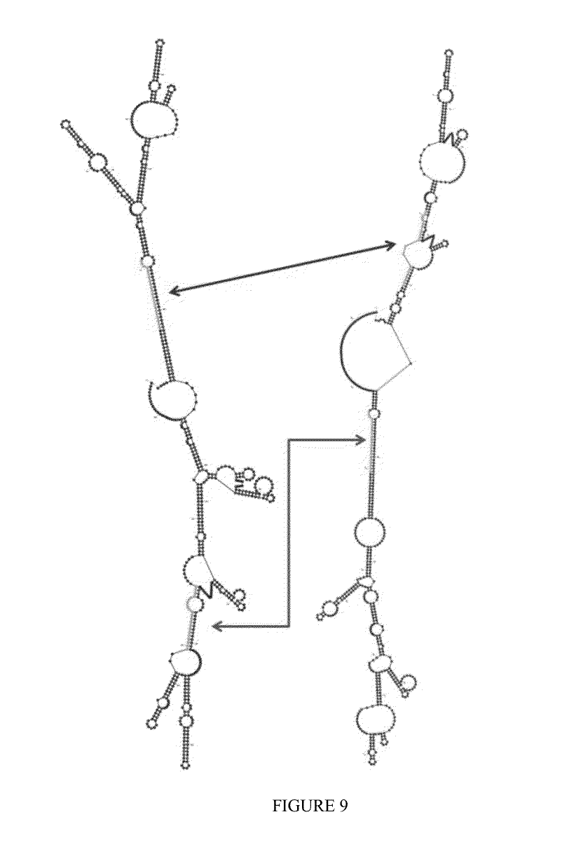

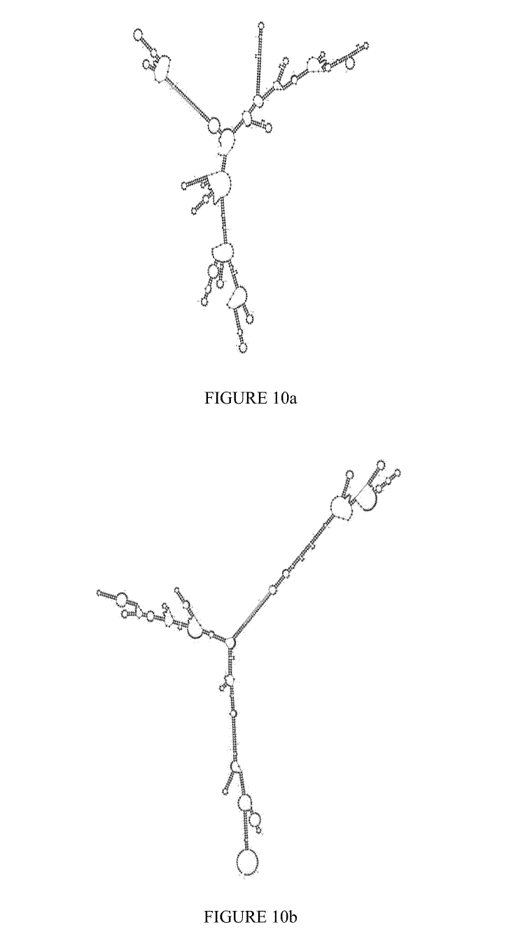

FIG. 9 is an image of the gymnosperm P. glauca predicted pri-miR828 transcript carries two miR828 sites on a polycistronic precursor (FIG. 10), while all analyzed dicot primiR828s have one (FIG. 9, data not shown).

FIG. 10a and FIG. 10b are images of the two predicted alternative secondary structures of P. glauca predicted pri-miR828 transcript with similar delta-G free energies form “good” hairpin structure which could generate mature miR828 from either of these candidate loci.

FIGS. 11a-11d are images of the evolution and function of MIR828 and TAS4 evidenced by sequence alignment and modified 5′-RACE validation of MYB endonucleolytic cleavage in a gymnosperm. FIG. 11a is an image of a sequence alignment for MIR828 genes from dicot, a basal monocot Trillium camschatcense, and gymnosperm (SEQ ID NOS: 66-70). Alignments were color-coded based on the confidence of the local alignment of T-Coffee (yellow\brown\red). The predicted mature miR828 sites are labeled with a black line. Asterisks show consensus (con) nucleotides identical for the given position. Abbreviations correspond to species listed as follows (with accession numbers from GenBank). Arabidopsis MIR828 sequences are from miRBase. In Picea glauca, since there are two miR828 sequences on one long precursor, sequences spanning the 5′ mature miR828 is used for alignment. At, A. thaliana; Aly, Arabidopsis lyrata; Tca, Trillium camschatcense (AB250300.1); Pgl, Picea glauca (CO236109.1); Pco, Pinus contorta (GT251244.1).

FIG. 11b is an image of an extended sequence alignment of candidate P. contorta miR828 gene and three predicted MYB targets from P. contorta (species) (SEQ ID NOS: 71-75). The GenBank accession numbers for MYB targets are shown. miR828 5′ arm (−) is the reverse complement sequence for the strand where mature miR828 locates. miR828 3′ arm (+) is the strand where miR828* maps. The location corresponding to mature miR828 is labeled by a black line.

FIG. 11c is an image of a sequence alignment for miR828 gene (SEQ ID NO: 76) and TAS4 (SEQ ID NO: 77) in A. thaliana showing homologies suggestive of a common evolutionary lineage. The miR828* site on the miR828 3′ arm (+) and the miR828 and siR81(−) complementary sites on TAS4 are indicated by black lines.

FIG. 11d is an image of 5′-RACE clone sequence data that established cleavage of a P. resinosa MYB target mRNA (SEQ ID NO: 78) by miR828 (SEQ ID NO: 79). All 14 clones sequenced mapped to the predicted miR828-cleavage site, based on the closely related P. contorta miR828. The candidate miR828s share significant similarity for mature miR828 and flanking regions, suggesting an ancient origin of miR828 (FIG. 11a). Interestingly, genomic sequences with great similarity to the miR828 orthologs were found in Trillium camschatcense (FIG. 11a “Tca”), a basal monocot species. The T. camschatcense miR828-like sequence would form an extensive hairpin (a hallmark of miRNA precursors) if expressed. In contrast to most monocots (which have characteristic narrow, thick, hard leaves with parallel venation and tiny, wind-dispersed seeds released from dry capsules), Trillium possesses broad, thin, soft leaves, net venation, and fleshy fruits. This phylogenetic relationship suggests a plausible hypothesis that miR828 was lost early in the monocot lineage and plays some important roles in gymnosperm and dicot physiology.

As shown in FIG. 9 the gymnosperm P. glauca predicted pri-miR828 transcript carries two miR828 sites on a polycistronic precursor, while all analyzed dicot primiR828s have one (FIG. 11a). FIG. 9 is an image of the two predicted alternative secondary structures with similar delta-G free energies form “good” hairpin structure which could generate mature miR828 from either of these candidate loci. Sequence comparison among miR828, MYBs and TAS4 revealed some clues for a monophyletic origin. By DNA sequence alignment, extended similarities were found across the reverse strand of the P. contorta 5′ arm of miR828 precursor, the sense strand of the 3′ arm, and three predicted cognate MYB targets (FIG. 11b black line). Similarly, when the A. thaliana TAS4 sequence is aligned with the arm for miR828* and its downstream sequences (presumably pri-miR828 sequence), they show extensive conservation, including and beyond the miR828 binding site (i.e. miR828*) and the region for the 3′ end of TAS4 (FIG. 11c). Our data suggests an inverted duplication model for the evolution of miR828 and TAS4. To search for evidence supporting our hypothesis for TAS4 origin, a modified 5′-RACE assay was performed on RNA samples from ancient land plants, including T. globosa and P. resinosa. Using the conserved nucleotide sequence footprint found within miR828 binding sites for TAS4 paralogs in dicot plants (FIG. 6), a degenerate primer was designed as described (Axtell and Bartel 2005). However, we were unsuccessful to clone any TAS4 sequences (data not shown). Interestingly, MYB-like genes were cloned from these studies which had plausible miR828 complementary sites (data not shown).

FIG. 11d is an image of modified 5′-RACE clones sequenced to establish cleavage of a P. resinosa MYB target mRNA by miR828. Consistent with our model, no remnant TAS4-siR81(−) complementary site was found within this MYB cDNA sequence (GenBank accession no. HQ997774). These data support the existence of miR828 and a regulatory role in gymnosperms.

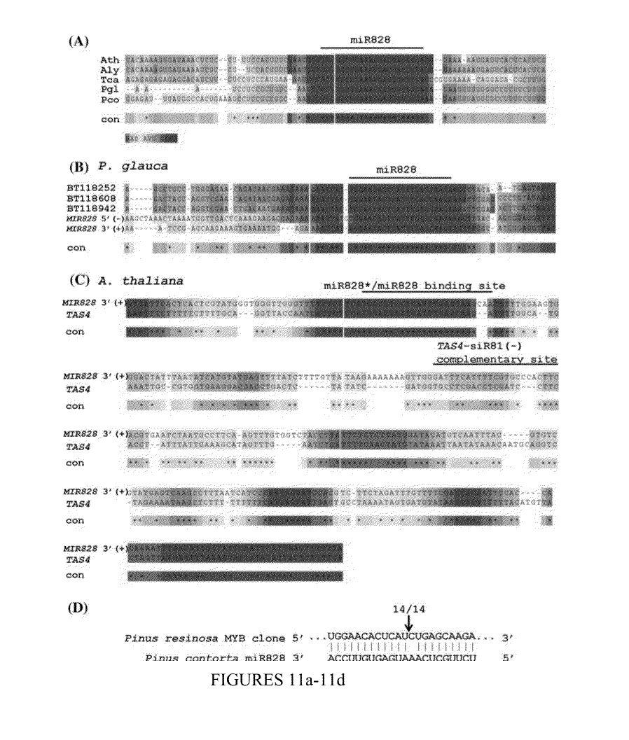

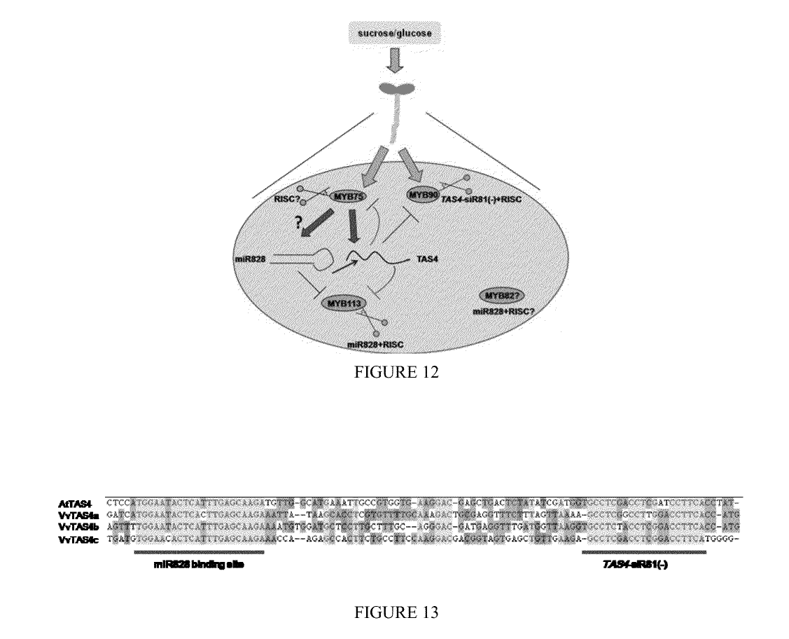

FIG. 12 is an image of the feedback regulatory loop involving PAP1/MYB75 and TAS4 in response to sugars in Arabidopsis. PAP1 expression is induced by sucrose, glucose or other stimulus. PAP1 may regulate TAS4 expression presumably by binding to the PAP1 cis regulatory elements in TAS4 promoter (FIG. 1a) and transactivate its transcription. Alternatively, TAS4 expression may directly respond to sugar stimulus through a signaling pathway involving PAP1. Increased TAS4 transcript abundance generates more TAS4-siR81(−) through the ta-siRNA pathway, which then down-regulates PAP1, PAP2 and MYB113 expression levels. miR828 controls MYB113 expression by guiding MYB113 transcripts into RISC. At the same time, miR828 also promotes TAS4 cleavage and routes its cleaved product into tasiRNA pathways for TAS4-siR81(−) biogenesis, which reinforces the feedback loop involving PAP1 and TAS4, as well as the regulatory network on PAP2 and MYB113 by TAS4. It is not clear whether PAP1 regulates miR828 transcription, or whether miR828 can down regulate the expression level of MYB82, a putative miR828 target.

PAP1 and TAS4 respond to endogenous sugar signals Based on the presented data, we propose a working model for the auto regulatory feedback loop involving PAP1 and TAS4 (FIG. 12). PAP1/MYB75 expression is induced by exogenous treatment of physiological concentrations of Sucrose and Glucose in Arabidopsis seedlings. Sucrose may be transported into the nucleus by Sucrose transporter(s), which activates Suc-induced transcription factors that bind to the promoter of PAP1 and activate its transcription (orange arrows). The elevated expression of PAP1 may bind to the promoter of TAS4 via PAP1 cis-elements and promote the transcription of TAS4. TAS4 may also respond to sugar stimulus through a signaling pathway in which PAP1 is involved. The subsequent increased expression of TAS4 will produce more TAS4-siR81(−) by the guidance of miR828 through RISC-mediated cleavage, which then reduces the PAP1 transcript level by the same mechanism. The proper regulation of PAP1 expression level by the auto regulatory feedback loop would give plants a means to monitor changes in nutrient and/or environmental conditions.

PAP1 cis-regulatory elements are also found in the putative promoters for miR828 and PAP1 itself (FIGS. 1b and 1c), one of which may locate within the 3′-UTR of FOREVER YOUNG (FEY, AT4G27760), a gene upstream of miR828 (AT4G27765). This could suggest a complex transcriptional regulation by PAP1 on TAS4, miR828, and itself Sugar sensing and signaling pathways have been tightly linked with Pi bioavailability in the root responding to Pi starvation. Arabidopsis plants accumulate starch and sugars in the leaves when treated with low Pi. Several phosphate starvation-responsive genes are sugar-inducible, including purple acid phosphatase 17 (PAP17/ACP5), ribonuclease 1 (RNS1), and induced by phosphate starvation 1 (IPS1). On the other hand, some hexokinase-independent sugar-sensing genes, for example β-AMYLASE (β-AMY) and chalcone synthase (CHS), are induced by Pi starvation in detached leaf assays as well (Muller et al. 2005). Interestingly, PAP1 expression is triggered by Sucrose treatment and Pi starvation to similar levels (4- and 3.5-fold, respectively) in a leaf transcriptome profiling study (Muller et al. 2007). miR828 and TAS4-siR81(−) expression respond to Pi deficiency in the shoots of Col-0 as shown by sRNA deep sequencing and Northern blot (Hsieh et al. 2009). However, this finding was not observed by other groups using either RT-PCR, sRNA sequencing, or locked nucleic acid-based microarrays. It has been shown that Sucrose synthesis increases in the leaves of Pi-deficient Arabidopsis, bean, barley, spinach and soybean plants, although some variation may exist. Sucrose in the shoot can also be translocated to the root via phloem as the causal intermediary signal, supported by the evidence that Sucrose concentrations in the root of Pi-starved soybean plants are higher than that in Pi-replete plants, but not in Arabidopsis. In addition, genetic screens identified a Pi-deficient mutant, PHO3, with reduced root acid phosphatase activity under low Pi conditions. PHO3 is allelic to SUC2, a Sucrose transporter for phloem loading. The PHO3 mutants accumulate high levels of Sucrose and other carbohydrates because of its inability to translocate them to the roots. Strikingly, PAP1 and PAP2 expression is significantly increased in pho3 mutants based on transcriptome profiling. Taken together, we propose that the upregulation of TAS4-siR81(−) and miR828 in Pi deficiency could be the consequence of accumulation of Sucrose and/or Glucose in the shoots. In line with this, TAS4-siR81(−) and miR828 are found in shoots, but not roots, of Col-0 seedlings under Pi starvation. Evolution of TAS and MIRNA genes. We mapped the cleavage site on a MYB target guided by miR828 in P. resinosa, providing direct evidence for miR828 function in gymnosperms. Although P. resinosa miR828 was not found in this study, its paralogs were predicted in closely related P. contorta and P. glauca species with the same mature miR828 (FIGS. 9-11a). It may indicate the conservation for miR828 sequence and for its regulation of MYB targets in gymnosperms. Interestingly, the regulation of MYB expression in dicots may be different from that in gymnosperms. PAP1/PAP2/MYB113 in Arabidopsis all carry TAS4-siR81(−) binding site, and MYB 113 is targeted by miR828 as well, which was confirmed by 5′-RACE.

PAP1 and/or PAP2 are expressed more abundantly and widely than MYB 113. For example, PAP1 expression is induced by a variety of stress conditions such as heat, drought, chilling, N deficiency, and ABA in addition to sugars, whereby anthocyanin is accumulated. The common availability of TAS4-siR81(−) binding sites in these MYBs could point out a more important role for TAS4 regulation of them in dicots. miR828 may function as an upstream riboregulator for MYBs, in which it fine-tunes TAS4 expression, whereas the downstream TAS4-derived siRNAs control MYB transcript levels. How miR828 and TAS4 coordinates MYB expression in response to different physiological conditions becomes a critical question to answer. Although the modes for generating ta-siRNAs and their functions in gene regulation and plant development have been extensively studied, little is known about the molecular evolution of TAS genes. miR828 and TAS4-siR81(−) regulate the same set of target genes indicates phylogenetic analysis.

From our bioinformatic approaches and RACE assays, TAS4 paralogs are only found in dicot plants, while miR828 and its target orthologs are extant in gymnosperms and dicotyledonous plants, suggesting a more ancient origin for miR828. The extended homologies of cognate miR828 with its targets in P. contorta, and for TAS4 and miR828 3′ arm with miR828* in Arabidopsis (FIGS. 11b and 11c) may provide hints for an evolution pathway from miR828 to TAS4. Subsequently, a duplication event may have occurred on the 3′ arm of miR828 to give two miR828* sequences. Such events could give birth to a proto TAS4 gene, which would be captured by the ta-siRNA pathway(s). Superimposed evolutionary constraints may have driven it towards a role as a regulator of MYB gene expression. From the miR828-like DNA sequence in T. camschatcense (FIG. 11a), we suggest miR828 sequences died early in the monocot lineage.

The present invention provides that VvTAS4a and its effector miR828 in grape can target novel MYB transcription factors as effectors of anthocyanin production and veraison in grape. Elucidation of the molecular mechanism by small RNAs of post-transcriptional control of veraison (the transition in grape berry development from acid to sugar accumulation, production of volatiles, and antioxidant pigments) provides the means to manipulate the process by RNA interference and transgenic technologies. Specific example include transgenic grape that expresses a TAS4-siR81(−) or miR828-resistant recombinant MYBA6, MYBA7, LOC100244963, LOC100247476, LOC100240800, and LOC100249722 genes.

FIG. 13 is an image of the sequence of the miR828 and ta-siR81(−) footprints show selective sweep in grapes with three functionally conserved TAS4 loci as seen in FIG. 13. The VvTAS4a locus is expressed in pre-veraison berries and flowers (SEQ ID NOS: 80-83).

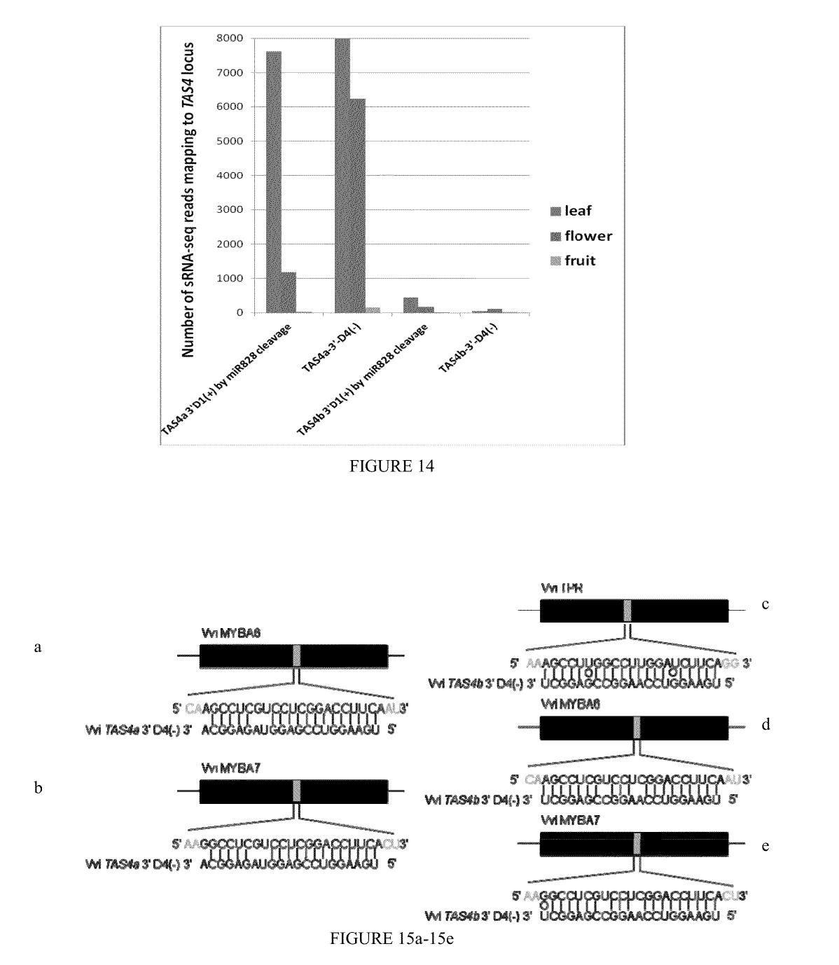

FIG. 14 is a graph of the expression of TAS4 genes in grape deduced from sRNA-seq read abundance showing TAS4ab are highly expressed in grape leaf and flower but not fruit and that miR828b is expressed in leaf (data not shown). Four grape MYBs have near perfect target sites for Vv-miR828 in grape mRNA.

FIG. 15a-15e is an image of the sequence that predicts siRNA81(−)/D4(−) from VvTAS4ab loci targets MYBA6, MYBA7 and an “off-target” TPR. None of these predicted sRNA target genes have been described in the literature to be effectors of grape berry development. TAS4 and its effector miR828 function to regulate grape berry anthocyanin biosynthesis and veraison. Since TAS4 and miR828 are not on the commercially available grape exon microarray, this discovery has been missed by other researchers in the field who are using empirical correlative approaches. Near perfect targets to Vv-miR828b (mismatch at nt 2) in grape genome: LOC100244963 uncharacterized MYB transcription factor, chromosome 17; LOC100247476 uncharacterized MYB transcription factor, chromosome 14. Mismatch at nt 17: LOC100240800 uncharacterized, unmapped MYB transcription factor on scaffold assembly NW_{—}002240894.1; LOC100249722 uncharacterized MYB transcription factor, chromosome 9. Near perfect targets to Vv-TAS4c-siR81(−) in grape genome (mismatch at nt 15): GSVIVT01030819001/VvMYBA7 uncharacterized MYB transcription factor, chromosome 14.; GSVIVT01030822001/VvMYBA6 uncharacterized MYB transcription factor, chromosome 14.

A trans-acting small interfering RNA (TAS4) and its cognate microRNA (miR828) targets MYB transcription factors involved in ABA and drought response and anthocyanin production in Arabidopsis. We have found miR828 homologs in ESTs from leafy spurge, spruce, poplar, cedar, morning glory, Mimulus (monkey flower), grape, Hedyotis (starviolet), lettuce, and ESTs homologous to its target TAS4 in poplar, grape, monkeyflower, leafy spurge, and salt cress (a stress-tolerant relative of Brassica and Arabidopsis). These findings support the hypothesis that the identified posttranscriptional mechanisms precede the monocot-dicot divergence and are under strong evolutionary pressure for adaptive stress-response processes.

The present invention provides production of stably transformed cotton and tobacco expressing Arabidopsis and TAS4:resistant MYBs by biolistic transformation of transient expression constructs and/or use of T-DNA vectors and phenotypic, physiological, and molecular characterization of transgenics that overexpress effector genes for ABA-, drought-, and salt-sensitivity/tolerance. The present application provides functional testing in plants for interaction between phospholipase D, JIM19, ABI1 and ABA receptor components and physical interaction studies. Functional analysis of MYB90/MYB75/MYB 113 (TAS4 targets) for ABA-inducible gene expression in Nicotiana benthamiana. Structure-function of TAS4 cis elements in MYBs. The present invention also provides molecular characterization of TAS4/miR828 gene and targets in gymnosperms and dicot crops. The expression levels of miR828, TAS4-siR81(−) and MYB75, MYB90 and MYB113 were assayed in response to salt, drought, and ABA. Modified 5′- and 3′-RACE will be performed on the TAS4 transcript (or MIR828 for gymnosperms) and MYB targets from gymnosperms and dicots to map 5′ and 3′ antisense transcript and validate TAS4- or miR828-mediated cleavage.

The present invention provides model plant Arabidopsis a sugar-, phosphate-, and ABA auto-regulatory loop involving a microRNA (miR828) and Trans-Acting-Small RNA locus4 (TAS4). In the overlapping plant RNA interference (RNAi) pathways that include miRNAs and TAS genes, 21-nucleotide duplexes of small interfering RNA (siRNA) are processed from longer double-stranded RNA precursors by the RNaseIII Dicer-Like 4 (DCL4). Single-stranded siRNAs then guide ARGONAUTE1 (AGO1) to execute posttranscriptional silencing of complementary target RNAs. We have shown that MIR828 and TAS4 are structurally and functionally related non-coding RNA loci which down-regulate anthocyanin biosynthesis in Arabidopsis by Watson-Crick base pairing to, and subsequent endonucleolytic cleavage of, transcripts of PAP1/MYB75, PAP2/MYB90, and/or MYB113. TAS transcripts are targeted by miRNAs and trigger distinct antisense transcription and feedback loops that amplify the production of siRNAs that in turn negatively regulate target gene expression in trans.

Golden kiwi (Actinidia chinensis), apple, poplar, Gerbera (an ornamental related to sunflower), Ipomoea (morning glory), petunia, monkey flower, and cocoa (Theobroma cacao), substantiates the claim that miR828 and TAS4 are conserved and functional in these species. Furthermore gymnosperms (several Pinus and Picea spp.) and possibly Trillium camschatcense (an ornamental plant) have functional MIR828 genes that target for silencing mRNAs encoding MYB transcription factors. There is a large body of evidence (reviewed in Hichri et al., 2011) that the clade of MYBs targeted by miR828 and TAS43′D4(−) siRNAs regulate phenylpropanoid pathways in plants important for secondary metabolite biosynthesis. The products of the phenylpropanoid pathways function in plant development and defense, contributing colors to fruits and flowers, and adaptation to environmental conditions such as cold or UV stresses and pathogen attack. These metabolites are substrates for biosynthesis of lignin (structural components of wood) and fruit and seed pigments, a source of anti-oxidants and neutraceuticals, and are therefore of agronomic and industrial value. Examples are provided of expressed (mRNA, therefore functional) sequences from bean, citrus, eucalyptus, Japanese cedar, (Cryptomeria japonica), chicory, Taraxacum kok-saghyz (Russian Dandelion or Rubber Root), and Gossypium spp. targeted by miR828 and possibly TAS43′D4(−)-like siRNAs. For Gossypium, the cognate miR828 was found in small RNA deep sequencing dataset from G. arboretum flower sRNAs.

For every example, it is shown bioinformatatically that the expressed sequences are either functionally conserved homologues of MIR828 or TAS4, where the collective body of evidence is that cognate mature miR828 or TAS43′D4(−) species have cognate MYB mRNA targets, and examples are provided of species-specific targets. The present invention claims a conserved regulatory control mechanism based on Watson-Crick complementarity and known topology for specificity of plant miRNA function as detailed by Fahlgren and Carrington (2010). Namely, the seed region (n.t. 12-20 of the mature miRNA reverse complement) must be perfectly complementary (with at most a single G:T wobble), and one or two mismatches at other positions (n.t. 1-11 of the queries below) in the miRNA.

The gram-negative bacterium Xylella fastidiosa (XF) infects numerous crops and is the single greatest threat to the long-term survivability of susceptible grape cultivars. Pierce's Disease (PD) is caused by XF, which is indigenous to the Gulf Coast region of the United States. Different races of this organism appear to be host specific, suggesting gene-for-gene host-pathogen interactions have evolved. Grapevines become infected when a sharpshooter insect that carries the bacterium feeds on tender tissue. The infection process involves the blockage of xylem vessels responsible for the passage of water, but published work shows that water deficit in grapes and other species does not produce the same symptoms as XF infection, for example leaf scorch. Two obvious symptoms of XF infection in grapes are shriveling of berries, and accumulation of anthocyanins in concentric zones of leaves. Phenolics also accumulate in xylem sap of infected almonds. These effects are also seen with nutrient (specifically Pi) deficiency and ABA/stress responses in grape and generally in plants.

XF infection results in significant and reproducible decreases in xylem [Pi]. The role of the plant stress hormone abscisic acid (ABA) in wilting and anthocyanin accumulation in plants is well established. In the context of XF disease symptoms of wilting and berry shrivel, it is interesting to note that vascular parenchyma cells at the boundary between xylem and phloem bundles are the site of drought-inducible ABA biosynthesis and transport by the action of nine-cis-epoxycaroteniod dioxygenases)/4 (VvNCED1/4), the rate-limiting step of ABA biosynthesis, is which is also up-regulated during berry development. Flavonoids are secondary metabolites involved in several aspects of plant development and defense, contributing colors to fruits and flowers that aid in seed and pollen dispersal, and adaptation to environmental conditions such as cold or UV stresses, and pathogen attack. Flavonoids, stilbenes, and polyphenolics are components of wine that impart color, aromas, and flavor enhancers associated with purported nutraceutical benefits (e.g. antioxidant and anti-inflammatory properties) and in-mouth tactile sensations (astringency). They comprise three classes of compounds whose biosynthesis during véraison and stress response is mediated by genes encoding enzymes for committed branch pathways: flavonols by flavonol synthase (VvFLS) and UDP-glucuronic acid/galactose:flavonol-3-Oglucuronosyltransferases (VvGT5/6), anthocyanins by UDPglucose: flavonoid 3-O-glucosyltransferases (VvUFGT), and proanthocyanidins by leucoanthocvanidin reductase (VvLAR) and anthocyanidin reductase (VvANR).

Recent reports have shown that sugars and sunlight induce anthocyanin accumulation and flavanone 3-hydroxylase (F3H, an upstream enzyme in the flavanoid pathway) in grape berries. Water deficit increases stilbene metabolism. Pre- and postvéraison water stress affects anthocyanin composition differently, suggesting a differential regulation of genes involved in the last steps of anthocyanin biosynthesis pathway. The molecular mechanisms that regulate veraison (the seed maturation phase in grape berry development that coincides with transition from acid to sugar accumulation, production of volatiles and antioxidant tannic pigments, and breakdown of chlorophyll and methoxypyrazines) are unknown, but the plant drought stress hormone ABA has been implicated. Transcriptional profiling studies in model species including grape have revealed that 8-10% of plant genomes are either induced or repressed by ABA at a single developmental stage. ABA is a small lipophilic sesquiterpenoid (C15) that plays important roles in plant growth, development, sugar signaling, and anthocyanin biosynthesis. Beyond abiotic stress adaptation and seed development, ABA has been implicated as playing a key role in pathogen virulence, which may provide insight into why many bio- and necrotrophic microbes including Botrytis cinerea have evolved the ability to synthesize ABA. The Botrytis “noble rot” of winegrapes, as opposed to the destructive “bunch rot” symptoms triggered by wounding, occurs when drier conditions follow wetter at veraison. The fungus causes desiccation of the grapes, leaving behind higher concentrations of sugars, fruit acids and minerals that impart distinctive properties to wines. There is mounting evidence that plant immunity to Botrytis, other microbial pathogens, and even arbuscular mycorrhizal symbioses and parasitism require RNA silencing pathways including microRNAs and trans-acting small interfering RNAs (miRNAs and ta-siRNAs), suggesting a broader role for plant small RNAs in environmental responses and evolutionary adaptation.

Pi starvation by PD leads to anthocyanin accumulation by down-regulation of miR828/TAS4 and crosstalk with ABA and sugar signaling. Phosphorous is the second most limiting nutrient to plants. Inorganic phosphate (Pi) deficiency causes delays in grape berry maturity and reduced berry size, but the molecular mechanisms are unknown. Sugar sensing and signaling pathways are tightly linked with Pi bioavailability in the root responding to Pi starvation. Arabidopsis plants accumulate starch and sugars in the leaves when treated with low Pi. Several Arabidopsis phosphate starvation-responsive genes are sugar- and ABA-inducible, whereas some hexokinaseindependent sugar-sensing genes, for example β-AMYLASE (β-AMY) and chalcone synthase (CHS), are induced by Pi starvation in detached leaf assays. Interestingly, production of anthocyanin pigment 1 (PAP1/MYB75) expression was induced to similar levels by sugar treatment and Pi starvation (four-fold) in a leaf transcriptome study.

Endogenous siRNAs pass through plasmodesmata and move across graft unions in phloem to regulate gene expression by epigenetic modifications. The novel target VvMYB genes are key effectors of anthocyanin accumulation in PD etiology and veraison, mediated through Pi sensing crosstalk that modulates miR828 and TAS4 activities which function normally to repress target MYB expression. FIG. 14 provides expression evidence that VvTAS4 dramatically decreases during grape berry development, supporting the hypothesis of miR828/TAS4 regulation may be the cause of anthocyanin accumulation in XF infections. A recent report validated miR828 cleavage of novel MYB transcripts in grape, and FIG. 15 provides bioinformatic evidence that a TAS4 antisense siRNA targets different novel VvMYB transcripts (MYBA6/7). All these novel MYBs are most homologous to those MYB transcription factors (viz, VvMYBA1/2/3, F1, PA1/2) that have been shown to be effectors, respectively, of Anthocyanin, Flavonol, and ProAnthocyanidin pathways in grape.

It is noteworthy that VvMYBA1/2/3 isogenes do not account for all observed anthocyanin phenotypes or flavonoid pathways. Association mapping demonstrated that only 62% of anthocyanin content variation could be attributed to the VvMYBA1/A2/A3 cluster of genes on chromosome 2 in a Syrah×Grenache F1 pseudo-testcross, consistent with our claim that other upstream regulatory genes, viz. the MYB genes are targets of miR828 and a TAS4-derived siRNA, control veraison and leaf pigmentation in response to XF. Evidence for VvMYB genes controlling fruit and leaf traits are the pleiotropic phenotypes of Pinot Meunier sport “Samtrot,” which has glabrous apices and leaves, a well-known phenotype caused by MYB mutations in many species, a lower fruit set, yields, and higher sugar content than compared to Pinot Meunier or Pinot noir. Additional support is the mapping of a quantitative trait locus for PD resistance very near to where predicted TAS4a3′D4(−) targets MYBA6/7 and a miR828 validated target GSVIVT00020733001 reside on Chr. 14.

Grape has three functionally conserved TAS4 loci (FIG. 13). The VvTAS4a locus is expressed (EC986896, CF609355) in pre-veraison berries and flowers. We have shown that sense and antisense siRNAs derived from TAS4ab are highly expressed in grape leaf and flower, but not fruit (FIG. 14), and that miR828 is expressed in leaf (data not shown). Five grape MYBs that have near-perfect target sites for Vv-miR828b are cleaved in planta.

As seen in FIGS. 15a-15e, siRNA81(−)/3′D4(−) from VvTAS4ab loci are predicted to target MYBA6 and MYBA7. None of these Vv-miR828 and predicted TAS43′-D4(−) target MYB genes have been described in the literature. TAS4 and its effector miR828 regulate PD disease symptoms and véraison as well as upstream steps (viz. PAL, CHS, CHI, F3′5′H, DFR, LDOX) in anthocyanin, flavonol, and proanthocyanidin synthesis.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine studyation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue study in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of regulating plant development and secondary metabolite biosynthesis comprising the steps of:
providing one or more plant cells;
providing an anthocyanin effector to the one or more plant cells, wherein the anthocyanin effector affects one or more transcription factor; and
regulating the expression of one or more genes using the one or more transcription factor, wherein the one or more genes affect the plant development and/or secondary metabolite biosynthesis.

2. The method of claim 1, wherein the one or more anthocyanin effectors comprise a small interfering RNAs (ta-siRNAs), a Trans-Acting SiRNA Gene 4 (TAS4), MIR828 or a combination thereof.

3. The method of claim 2, wherein the small interfering RNAs (ta-siRNAs) comprises TAS4-siRNA81(−).

4. The method of claim 1, wherein one or more anthocyanin effectors comprise a MIR828 that triggers the cleavage of a Trans-Acting SiRNA Gene 4 (TAS4) transcript to produce a small interfering RNAs (ta-siRNAs).

5. The method of claim 1, wherein the one or more anthocyanin effectors comprises a construct coding for a TAS4.

6. The method of claim 1, wherein the transcription factor comprises a set of MYB transcription factors selected from PAP1, PAP2, MYB 113 or a combination thereof.

7. The method of claim 1, wherein the one or more anthocyanin effectors comprises a VvTAS4a that affects a MIR828 to trigger the cleavage of a Trans-Acting SiRNA Gene 4 (TAS4) transcript to produce a small interfering RNAs (ta-siRNAs).

8. The method of claim 1, further comprising the step of providing a miR828 to the plant cell, wherein the miR828 triggers the cleavage of a Trans-Acting SiRNA Gene 4 (TAS4) transcript to produce a small interfering RNAs (ta-siRNAs).

9. The method of claim 1, wherein the anthocyanin production and veraison affects Pierce's Disease.

10. The method of claim 1, wherein the one or more plant cells comprise a berry cell, a bean cell, a citrus cell, a eucalyptus cell, a Japanese cedar cell, a chicory cell, a Russian Dandelion or Rubber Root cell, and combinations thereof.

11. The method of claim 1, wherein the one or more plant cells comprise grape cells, golden kiwi cells, apple cells, poplar cells, Gerbera cells, sunflower cells, Ipomoea cells, petunia cells, monkey flower cells, cotton cells, and cocoa cells.

12. A composition to manipulate one or more plant properties comprising:
a carrier comprising a construct that codes for a interfering RNAs (ta-siRNAs), wherein the interfering RNAs (ta-siRNAs) targets a transcription factor to affect the anthocyanin biosynthesis pathway to affect one or more plant properties.

13. The composition of claim 12, wherein the construct comprises a DNA or a RNA.

14. The composition of claim 12, wherein the construct comprises a double-stranded RNA is cleaved to form siRNA, a double-stranded DNA that codes for an interfering RNAs (ta-siRNAs), or a combination thereof.

15. The composition of claim 12, wherein the construct codes for a miR828 microRNA wherein the miR828 microRNA triggers the cleavage of a Trans-Acting SiRNA Gene 4 (TAS4) transcript to produce a small interfering RNAs (ta-siRNAs).

16. The composition of claim 12, wherein the small interfering RNAs (ta-siRNAs) comprises TAS4-siRNA81(−).

17. The composition of claim 12, wherein the transcription factor comprises a set of MYB transcription factors.

18. The composition of claim 12, wherein the set of MYB transcription factors comprises PAP1, PAP2, MYB 113 or a combination thereof.

19. The composition of claim 12, wherein the one or more plant cells comprise grape cells.

20. A plant cell having one or more constructs to manipulate one or more plant properties comprising:
a construct that codes for a small interfering RNAs (ta-siRNAs), a Trans-Acting SiRNA Gene 4 (TAS4), MIR828 or a combination thereof that targets a transcription factor to affect one or more genes involved in a biosynthesis pathway to affect one or more plant properties.

21. A method of treating one or more symptoms associated with Pierce's Disease in plants comprising the steps of:
providing one or more plant cells having one or more symptom of Pierce's Disease;
providing an anthocyanin effector to the one or more plant cells, wherein the anthocyanin effector affects one or more transcription factor that target one or more genes; and
regulating the expression of the one or more genes to ameliorate at least one of the one or more symptom of Pierce's Disease.


SEQUENCE LISTING (84 sequences)

SEQ ID NO: 1 - DNA, 3882 bp, Arabidopsis thaliana
attcgtagttagttacttgtatttttcaacgtgctgcttaagcctaactaactacgaataaaacgtgttgataatcgtgttaaaattttctggtaatgttctactagtaccaatattatttgagaagtacatttagaaaaaatgaattcgaaaatgtaacatgttacaaaatttgcattgaaagaacaaaaaaagaaaagaaagatcattttcagtaaaagtgtatcacgatttcccccctactattgatataagcaagcaagtttttgttttatagtactagtcatataaccaaacaaatttatagttcgatttatttcatcatttcacattggattagttttcgaaaaacgtgaacatcgtcttaacacaatttttgtaagagggcacgtaacatcgttgtaacacaattgaactgcttaagaccgagtagtgttttcaaaatccacaacactaaaagattaaaagttaaccattgcttaattatggccgactgtttcggttcttgtctaatcttctctttccacaatcaacttgatttgtttttttttttttttcaatattgatgtttatcttttaataaagtattcttaacagcttagtattcatttcatttttatattatatagttatattctatgtaatgttatagtatgaatttgtaagccgttttccaattatcaaaaaaaaaaaagaacctaatttttgaaaggccatgttcattgccgcgtggatggttccgttgccatcaacattctaacatttaatatttggtagatttattcccaaaataaataaaataaaatcattttttttttcttaaaataaaatcattttacatacctttttggttagaacttttattgtgtagttgcaatcaaacagagttgtttattttgtttttgttccacctgccaaaaaaaaattgtttgttttaattatatcatcaacaaagattcccaccctctacaaattttattctgataaaactttttatctgaatgacaaaaactgtggagagcttggagatttatgtaaaatagacagtacactatatattgaggaaagaaaagttataaacatgctcaatgctagctaggatcatcagtcaattgaataccatgaatgatccagctacctacgttttggtagttgaaaacctagaaaatagagtcttccatcaaattcttgaaaatccgtgatacatatatagcaaactttctcccaaaacaagagtcgaatcaacttaaacacgaaaatctttttccaatcgattaatcttctcatgcaaagacatccacattattttgaaaagcaataagaatctcaagtcattttgactccatgtgttgaagacgtgattcatttccataagaatgcataacaaatcttctccaactcttcaagatatatatgtttaggatcggagtaggaagcttatattgaaacaatgaaactccaaaactaaagtgtgttattatgaatcacggattggataaaacaaaatgaaatgcagattaatgaaaaacaatattttgtatatgcttattagctacataaccaaatttatatagcaaaattaatatgaaaacaattgtgacaaaaaccgtaaataaatgatatataatacaagtaatttaaatgaatagtgtaatattattttacgataaatgtaccaagttatttacgatttgatagccactgttttgtgaataaaagagagcatattcgcaattaggttttctatttgatcaagtgggaccttaaatagtcaaaattaaataaatcactattcaaaaagcacaagtgaaagatatttccgtggctggagagtaatgcgtgacatttggagtggaagcatctgatagcgagatggttgcgttgaaatccccaacaaccatccaagcaaaaagctcttgcgatgctttttaatatgcgtttaaattcttttttttttttaaataagaaatatatatagagttgagatattaaatggtgagtatatccaccactccacgtgaaacttgatcaatttaatcattcataactttgaactttgaactttgaaggggagttgtcatagatttgatgcttctacaactcttttttgtacttgtaagtggaactggaacccatatatcgtcttgttaatatcataacgtagtgtaaatggaacaatatggagtttcaaaagctacttaacacaagcatgttgctttgaatatatttattttggtgaaattatttagggataattaaggttttggtttttggtcggtttatttcgatttttaatatgtttggttcggtttagttttaaaatcaaaccaagtagatgttccggcgtgaattaattcattctattgtttgatgcatgtgattgattttcacgatatacggatccttttttgtaacttttgtttaatagtattatttggccggtataattactaagaatctatgtcgctttttattgattcgatgtttttgacttaatattatgatcagtaattataacattctaaattacaataaataactacaaagtcattccaaaatttataccatatggtatatgataactcagctacatttaagttaggttttggtttggtttgagattttatgaaacaacccatttatatttattttttgttttcctttctatgtagtagaaccatcaagtaatcaattacgtacgtcaccatacttaagaaacagtatcgcgcgatcgtgatggttatatacatatattatatatgtgtgcatgcaggcccggcttagagggtgtgcccttggtgctagtgcacagggtcctttattttttcattcattttctttaagcaatcaaggtccattatacaaacagtagtgtataataaactaaaatctaaacatataaaacatatattagaaaaaaaaactaatgagcacagggtcccacgttttgttgagccggccctgagtgcatgcatttcacatctacgtacatgtgtatatatgctgcatgcatacgcgcgcaacaaacgtgtaggctcgagagtcaagactgctaagtaagcactcatcacatgcacgttttacataataacccgtcaaactatcacacaagatgaattaagcatatactaatagattgtgggggcttgaaatcgaattctgttaattggggtatatgtatattaattactacgaatatatggcgtcgtcttttttacgattagtctaaacttgatacctgaatggaacttgcttacatatctgtgtatctatccatatataattcaaaattttaatattttgggtgaaattttttatgacccaaaaaaatacaaaacgcaagaagcgtgtaataaccaaattctcaaaaccacaagcccagccttaaaagacttgttatacatgaaaatatcaaatatttatgtatgttcaaggccggcactgcagcatacatcgcatatgtgattccatccattttgtttgacgatctttttttttttttcttaaagaaaaaatctttcgaataaacctacaaattaactaacattttctcatcaaaaaatttgatttatatacaaagaaattgtgaattgatagcatgagaacatactatgttatagtatatgattgtgattagacataaccgctgacaatacaatgagtataactgtataaggtatattgccaatttagtattaaaaaatgttggaggttagagcatatatatcccaaccaccaaaacaaataaaagacatttatatgtctaattctctactaccttagatttaccccaattgttatttcctaccaaacacgtatatacatacacatgtatgtaagctttactttgtctatttaaatagagtctacttgatcattttt

SEQ ID NO: 2 - DNA, 2070 bp, Arabidopsis thaliana
tcaacgctcggttaggacctttgcatgttctgattaacaatgctgggatgtttgctatgggaggttggttttattgccgttttagctataagcattgagtttccccttctgttgtttcctgtatctattttcatcatctctttgttatgatctcaaagtcatacttgtgaaccaatcatcatcatcatcaacattccaacttgatggacccaattggtgagaactggcatgttctttcatagcaactctgtccaatgggtctcctttttaaatgattggttgtcagatgctacattttctgcagtatgtgttgaggaaaagtaaccttaaaatgctctgttctctgatttgtgcagaggcgcaaaaattctcagaggaaggatatgagcagcacatgcaagtgaatcatttagctccagcgctgctttcagtacttcttttgccgtctctgatccgaggctctcctagccgaatcattaatgtgaattccgttgtaagttctctatgatcactgatactgcaggctccaagtctgtattctctagtttatattctctttggtatgagtaatcatgttgacaggttgatttgtttgtgtattctgtaacttgaaaacctctggtcttgtcaattggttgcagaatatgtagataactgatatagatctctctaaatcactgaactacatatgcatattgtattaacacagtcttggtatagagaaacgatacaaccatagcttgttgaaagtttcaaccccaaatacgcttccatgactgcactatgtcattactcatttcagaagaattctgtgcctggctgtttatgaaaactgaaacagtttatatttattgtctttgattattactttttcatgcaagataatgggttgtgggattcacttttgtagatgcatagtgtcggttttgttgacccggatgacatgaatgttgtttctggtagacgtaagtactcaagccttataggatactcaagcagcaagcttgcccaggtgagcttacagtaatgacatctgccgtagttttcatctgccgtagtttccattttctgaaagaggcttattttcagaaactggtgagttaagtctcgctctctgatcttccatcttccagattatgtttagtagcattcttttcaaaaagcttcctctggaaacaggagtcagcgtcgtatgtctatcccctggtgttgtcctaacaaatgttgtgagtattagcttcacttcatttcgttacaagtgcaggattcctctgttttctcagaaattcattgcgttgaactgcctatttctttccgaaatttacaggccagggatctatccaggattcttcaagctctttacgcagtgataccttatttcatattttcaccccaagaaggttgtagaagttctctattctcggccacagatcctcagattccagagtactgggaaacactaaaaaacgatgattggcctgtttgcccattcatctctcaagattgccgccctgcaaatccttccgaagaagcacacaacacagaaactgcacagagagtgtggaaaaagacgttagagctggtgggtcttcctctcgatgcagttgagaagctcatagaaggggaaaatatccaatgccggtatggagcacaacacgaatagtctttcaaaattaccacaggttaagtgacccattacagatcaaagggtaggtaattgagaaaatatcttttttttttgtttccttgtattaatctacacgatacagtggggaatgaatcccccaggcatgtagtttgcttgagaatgtttgattgttggataaaagtcaagctttagctaccattccattgcttttacttacaagtcttgttcaagtatttggttagtgtgtcttggagttattaagatgttcagtagtagagtatggtagcctgggttggtgggtagtttcttgtttgagtttagggtattttcgtaattttctttattgtttgtggagtcacgttgctcatatatagttatagggatatatatcaattagagtcatcatctctcttactctcatg

SEQ ID NO: 3 - DNA, 2750 bp, Arabidopsis thaliana
aaaatgatctggatgtgtttggttatatatataagaagacttaaaagataatgacttaatctcgtaacgagtcacacggacgtgacgctgaaactcacacacgttggtgccacgtctttgtctttcctcttttgctctacttttttctcctcataggtgataggtcccataagcaatgaaataaaaaaaatggtaattgacttttctccaaacattttcgaatctgattttctttttcaaggttttataacctctacattccagaatatgactaatgacatcattatccaattattttttatactgtaaactcattattatgaatattctttatttcaaaaaattaccattgatttataagtttattagtataatatataacatatggaataaaacttttatttaaaaaaaaatatttttccccaaaaaaagtaggattaataacctgattaataaataaaaagtgttatatttttaagcattgtatgcatttactttatcatagttgtcttgtttttaagagttaaaaaataatgatgaacaatttcacggacaacgattccacgataaagctttccctgcaacactcagattttctaaagacggttttgcattgcgttttctgggattcgaaacccaaacatgatgtacaagtattaatgaactcttagttaaccattagattaaaaatattttcactattaattttctcttaaaaatattaataattttttgaaatcaaaaattatagttattttattttaataaacgagaaacactacaaaaaaagttaactgcatttagataatttaataaactaaaatatccacataaaaatttcaaatttatcaaaaataaaacatcaatttgttttttgttttaaattaaagatttgctattgattgcataaggaagaaaactttacaaagccgaaaggcctaagagcccaacacacacaaaagaagaaccattttggatcaagggaaccgaccatgggtattagaagtagtggtataaagcccatcatatcccaacacataacccacgaatgtttaatattaaaagtttgttgttcggctcatgattagcgatgatcatacagaaagtttgtatctaatacgtgccttgaattttatgtgtacaacaaacaaattaaattattcaaaaccataaattataaaaaataattacagaaataaaactatattaagagcgagcctaccatccggtgtgcaactttctagtttatatacagtggcggatcaacgttaatgaggcaaattggttcaaattcatctaaataagactagagttcacaggttcgattcctccttataacaatttgctcccaccaattttttttgctgggtccgcccctggttatatatatacttctacaccaggtttgggttcgagtccacacataattaacgacacaattatagtgcacgatagaatgaactaaaacagctagagcgtagagggctcattgtctataaaaatccttcgttaacttgcaagaaaccaagagtagagggctcacacttaagtctcctacatgacgattatatttcgtcaaaaagaagcaattagttagctttacagcatatcatttcgcctaggttttccatcgtacacgtaaattttcatgcaagaaagcagaaatatacaaatactaacttttagatactgaaaaatgagatcagattctagtcaaattttgttaaaagtatttataaatttaaattgcaagtcctcaaaaagtacgactaaaaatgcttttcttagaaaatgataataaaccggcgttttatatataagtgtttctttttctcttctgtccagaagtaaatcattaagaaccaatatggcttttcttaaactaatctccgtgataatcaaatctttgatcattctccacacaatcccatcaacaacatcgatctcactagatgcaccaacaatgattctaatcggcactactaactatagagatagttgtcccaaaaaaaaaaaaaaaaactaactagagagataaatcatattcaatacatgtactatttctactatacttaagaaaatttgtataccactatcttaactcttaacactgaacatactatacactatcttaactcccaactcttgtaaaagaatatctaattttaagaaaagacttcaaatgcttgttaaatttctagtgaagatgcacattctaaaaactggtaaaatggtaagaaaaaaatatataaaaaaatagccttattaaaatttatatctcctatttctctatccaaactacacggatgaagcttattgttattcatccaccctttttctcaattctgtcctatttcttgtgcatgaaacttctccatcttgtaatcggataaatcatacccaaattttttctttctgaaaacatatatacccgaacattaattactatcgtcctttctcctaattttgttaagaaacatgtttgtttgtttttagtactgaaaaaggatggagatacttgctagatcctatgaaccttttctctctaggacaaatcagtaaccaaacaataacttagcaaattaagcacgacagctaatacataaaatgtggatatcaaacatgcacgtcacttccttttttccgtcacgtgtttttataaattttctcacatactcacactctctataagacctccaatcatttgtgaaaccatactatatataccctcttccttgaccaatttactt

SEQ ID NO: 4 - DNA, 22 bp, Artificial Sequence
  Synthetic primer
ggaatggttaaggctggattcg

SEQ ID NO: 5 - DNA, 22 bp, Artificial Sequence
  Synthetic primer
acgcatctttctgattcatccc

SEQ ID NO: 6 - DNA, 20 bp, Artificial Sequence
  Synthetic primer
ggttgaactatttgaagcca

SEQ ID NO: 7 - DNA, 22 bp, Artificial Sequence
  Synthetic primer
gcaattaaagaccacctattcc

SEQ ID NO: 8 - DNA, 20 bp, Artificial Sequence
  Synthetic primer
caaagggttgaggaaaggtg

SEQ ID NO: 9 - DNA, 20 bp, Artificial Sequence
  Synthetic primer
atcgatttagcccagctctc

SEQ ID NO: 10 - DNA, 20 bp, Artificial Sequence
  Synthetic primer
tcttggaaacagatggtcgt

SEQ ID NO: 11 - DNA, 21 bp, Artificial Sequence
  Synthetic primer
ttctgtcttcgggaattaggt

SEQ ID NO: 12 - DNA, 19 bp, Artificial Sequence
  Synthetic primer
cgacctcgatccttcacct

SEQ ID NO: 13 - DNA, 24 bp, Artificial Sequence
  Synthetic primer
attttctagacctgcattgtttat

SEQ ID NO: 14 - DNA, 21 bp, Artificial Sequence
  Synthetic primer
tgcctcgacctcgatccttca

SEQ ID NO: 15 - DNA, 21 bp, Artificial Sequence
  Synthetic primer
tggcatacagggagccaggca

SEQ ID NO: 16 - DNA, 22 bp, Artificial Sequence
  Synthetic primer
aggacttcccaggaggtcaccc

SEQ ID NO: 17 - DNA, 18 bp, Artificial Sequence
  Synthetic primer
tcgatttgtgggtgtcat

SEQ ID NO: 18 - DNA, 21 bp, Artificial Sequence
  Synthetic primer
atggagggttcgtccaaaggg

SEQ ID NO: 19 - DNA, 21 bp, Artificial Sequence
  Synthetic primer
atcaaatttcacagtctctcc

SEQ ID NO: 20 - DNA, 27 bp, Artificial Sequence
  Synthetic primer
cattgcgctctgattgtttgtttgtaa

SEQ ID NO: 21 - DNA, 22 bp, Artificial Sequence
  Synthetic primer
cacaagtaggccgcacgatgag

SEQ ID NO: 22 - DNA, 24 bp, Artificial Sequence
  Synthetic primer
tcgactaccattgaaggggattga

SEQ ID NO: 23 - DNA, 23 bp, Artificial Sequence
  Synthetic primer
ccattgaaggggattgagtgtcg

SEQ ID NO: 24 - DNA, 22 bp, Artificial Sequence
  Synthetic primer
tcttgctcaartgagtrytmca

SEQ ID NO: 25 - RNA, 21 nt, Artificial Sequence
  Synthetic primer
agccucgaccucgauccuuca

SEQ ID NO: 26 - RNA, 21 nt, Artificial Sequence
  Synthetic primer
acggagcuggagcuaggaagu

SEQ ID NO: 27 - RNA, 21 nt, Artificial Sequence
  Synthetic primer
agccucgaccucgauccuucu

SEQ ID NO: 28 - DNA, 205 bp, Arabidopsis thaliana
acatataaacctttttaagtttctttttcttttgcaggttaccaatcactctccatggaatactcatttgagcaagatgttggcatgaaattgccgtggtgaaggacgagctgactctatatcgatggtgcctcgacctcgatccttcacctatttattgaaagcatagtttgaatctcattttcaactatgtataaattaatat

SEQ ID NO: 29 - DNA, 200 bp, Theobroma cacao
ataaaagatcagatttggttctctgtgcagggaaggaatgtataagaaatctgtatggaacactcatttgagcaagaagcagctagttacttgtcttgtgcaaaggtggtgaaagttagatggctggttaacctcggcctcggaccttagtcggaaggtctccccatgtaggagtagcttctcgctattattgcacaagt

SEQ ID NO: 30 - DNA, 209 bp, Euphorbia esula
acttctttatttcaacatgaggaagttacttaagttggattgtgaagcatatatacaattggaacactcatttgagcaagagaccatatgtcactaatattttgagggcaccgatagcctacatggcttgactgcctcgacctcggaccttcattaccaatttgaaggtctttgccattattgtcactcaaatttttatacatatgtga

SEQ ID NO: 31 - DNA, 200 bp, Populus tremula x Populus tremuloides
agaaaacaaaaaaaccagggaagaatgacactgagacactgcatagcaaatattgtggaatactcatttgagcaagaaaatattagatacttatgttcaacgaaggtggaaaagctggatagcttggaagcctcgacctcgaaacttcaccattgattccaatggtgaaaagaaagatttctcattttattggataaaat

SEQ ID NO: 32 - DNA, 200 bp, Malus domestica
taaaaggaagtatgccgtctcaaactcagcaaagaactcgacagtaaattcacctgtagcactcatttgagcaagaatacacaactacgtttaataggcgaaggtcgccatctgctgccccgtgaaggaacctcaaccacgaaccttcacgaggggccaggagatgtatatgtttggccttctccaattaatatactaag

SEQ ID NO: 33 - DNA, 207 bp, Actinidia chinensis
gtcccggaatccgaaaattgggacagggatgtgaagagctttgcctaaaacatcacatggaatactcatttgagcaagataactagtataagttatattttataaggaggtagaggttagatggtcttagggcctcaacctcggaccttcagtgtaactgggatatgggtctaagagcgtaactacacgcaaggtaggatttaatgt

SEQ ID NO: 34 - DNA, 205 bp, Mimulus guttatus
aaccattgatttcatcgtcctacgtcgaggagaagaggttgtagtaagatttgtgtggaacactcatttgagcaagatatatttgtcggctaacacgatgatggcacgagggttatggggttttcgagtacctcgacctcggaccttcatctgtttttaatttatgtagtttttatttaataaagatgttagtagatacactcta

SEQ ID NO: 35 - DNA, 200 bp, Vitis vinifera
gaaaaaacagaatcagaaaatggatatgaacaaccatcttatgagatcatggaatactcacttgagcaagaaattataagcacctcgtgttttgcaaagactgcgaggtttctttagttaaaagcctcggccttggaccttcaccatggccaaaaggtggtgaggacctttctcaaacgtaataatatttgaggctgagg

SEQ ID NO: 36 - protein, 43 aa, Arabidopsis thaliana
LLGNRWSLIAGRLPGRTANDVKNYWNTHLSKKHEPCCKIKMKK

SEQ ID NO: 37 - protein, 44 aa, Arabidopsis thaliana
LLGNRWSLIAGRLPGRTANDVKNYWNTHLSKKHESSCCKSKMKK

SEQ ID NO: 38 - protein, 44 aa, Arabidopsis thaliana
LLGNRWSLIAGRLPGRTANDVKNYWNTHLSKKHDERCCKTKMIN

SEQ ID NO: 39 - protein, 44 aa, Vitis vinifera
LLGNRWALIAGRLPGRTSNDVKNYWNTHLSKKFFGKAVKDVADK

SEQ ID NO: 40 - protein, 41 aa, Gerbera hybrida
LLGNRWSLIAGRIPGRTANDVKNWWNTHLRSRHQQQQKVHQ

SEQ ID NO: 41 - protein, 41 aa, Ipomoea
LLGNRWSLIAGRIPGRTANDVKNFWNTHLQKKVSAMASSRQ

SEQ ID NO: 42 - protein, 41 aa, Petunia integrifolia
LLGNRWSLIAGRLPGRTANDVKNYWNTHLRKKLIAPHDQKQ

SEQ ID NO: 43 - protein, 44 aa, Glycine albicans
LLGNRWSLIAGRLPGRTDNEIKNYWNTHLSKKLRNQGTDPKTHD

SEQ ID NO: 44 - protein, 44 aa, Fagopyrum esculentum
LLGNRWSLIAGRIPGRTDNEIKNYWNTHLSKKLISQGIDPRTHK

SEQ ID NO: 45 - protein, 44 aa, Hordeum
LLGNRWSAIAARLPGRTDNEIKNVWHTHLKKRLDPSAQEQQEEA

SEQ ID NO: 46 - protein, 44 aa, Oryza
LLGNRWSLIAGRLPGRTDNEIKNYWNSHLSKKLIAQGIDPRTHK

SEQ ID NO: 47 - protein, 42 aa, Sorghum
MLGNKWSKIASCLPGRTDNEIKNVWNTHLKKPVFPACGWERT

SEQ ID NO: 48 - protein, 44 aa, Triticum aestivum
LLGNRWSAIAARLPGRTDNEIKNVWHTHLKKRLDPGAQEQLEEA

SEQ ID NO: 49 - protein, 39 aa, Zea perennis
LLGNKWSKIAACLPGRTDNEIKNVWNTHLKKKVAQREKK

SEQ ID NO: 50 - protein, 34 aa, Artificial Sequence
  Synthetic peptide
LLGNRWSLIAGRLPGRTDNDIKNYWNTHLSKKLK

SEQ ID NO: 51 - protein, 38 aa, Arabidopsis thaliana
RDITPIPTTPALKNNVYKPRPRSFTVNNDCNHLNAPPK

SEQ ID NO: 52 - protein, 38 aa, Arabidopsis thaliana
KNIISPPTTPVQKIGVFKPRPRSFSVNNGCSHLNGLPE

SEQ ID NO: 53 - protein, 38 aa, Arabidopsis thaliana
KNITSHPTSSAQKIDVLKPRPRSFSDKNSCNDVNILPK

SEQ ID NO: 54 - protein, 28 aa, Vitis vinifera
TAKVTVIKPRPRTFNKNLSYLKGKAAKE

SEQ ID NO: 55 - protein, 34 aa, Gerbera hybrida
EDELSQDTTVAIIKPQPRTFSKTLNWFGNRQSVK

SEQ ID NO: 56 - protein, 38 aa, Ipomoea
DSYWKGKAPEITENTVVRPRPRRFLKASSSPTTLLTEN

SEQ ID NO: 57 - protein, 36 aa, Petunia integrifolia
ESKNKAMKITENSIIKPRPRTFSRPAMNHVSCWNGK

SEQ ID NO: 58 - protein, 43 aa, Glycine albicans
KLTEAPEKKKGKKKNKQKNENNKGSEKTLVYLPKPIRVKALSS

SEQ ID NO: 59 - protein, 39 aa, Fagopyrum esculentum
PLSSAPNPNHGVVQTPKIADTPSTQKSFTFDPIRNPNAN

SEQ ID NO: 60 - protein, 42 aa, Hordeum
GAAKKRKKPAAAAAAPRKRDGKVKMRNLNALTARAAAAAPVS

SEQ ID NO: 61 - protein, 31 aa, Oryza
PLTAAADHSNAAAAVAATSYKKAVPAKPPRT

SEQ ID NO: 62 - skipped

SEQ ID NO: 63 - protein, 39 aa, Triticum aestivum
SAAKKRKKPAAAARKREGKIKMRKLDALTAKAAPVLSSP

SEQ ID NO: 64 - protein, 47 aa, Zea perennis
KAGAGSGDAGTPATAPLSSATSSTTTHNSSGGSDSGDQCGTSREPDA

SEQ ID NO: 65 - protein, 8 aa, Artificial Sequence
  Synthetic peptide
VKPRPRTF

SEQ ID NO: 66 - RNA, 84 nt, Artificial Sequence
  Synthetic primer
cacaaaagugauaaacucuccuuuccacuuucuaauguuucuugcuuaaaugaguauuccauguaaaaaugauucacucacucg

SEQ ID NO: 67 - RNA, 84 nt, Artificial Sequence
  Synthetic primer
cacaaaagugauaaaaucuccuuccacuuucuaauguuucuugcuuaaaugaguauuccauguaaaaaaugagucacucacuca

SEQ ID NO: 68 - RNA, 85 nt, Artificial Sequence
  Synthetic primer
agagagagagaggacaucuucuccucccaugaaaaugccucuugcucaaaugaguguuccauccgugaaaacaggagacgcuuug

SEQ ID NO: 69 - RNA, 66 nt, Artificial Sequence
  Synthetic primer
aaauccuccgcugucaaguuucuugcucaaaugaguguuccacauaauuuuuuguccucuucuuug

SEQ ID NO: 70 - RNA, 84 nt, Artificial Sequence
  Synthetic primer
ggagauuuauggccacugaaauccuccgccgucaagcuucuugcucaaaugaguguuccaaauaauuuauuuuccuuuucuuug

SEQ ID NO: 71 - DNA, 77 bp, Artificial Sequence
  Synthetic primer
aggctgcctgggagaacagacaacgaataagaattattggaatactcatttgagcaagaagcttacaatgagtagcc

SEQ ID NO: 72 - DNA, 82 bp, Artificial Sequence
  Synthetic primer
agactaccaggtcgaacagataatgagataaaaaactactggaatactcatttgaggaagaaaattggagccctgtataaac

SEQ ID NO: 73 - DNA, 82 bp, Artificial Sequence
  Synthetic primer
agactaccaggtcgaactgacaatgaaataaaaaactactggaatactcatttgaggaagaagattggagacctgactaaac

SEQ ID NO: 74 - DNA, 88 bp, Artificial Sequence
  Synthetic primer
aagctaaactaaaatggttgactcaaagaagaggacaaaaaattatgtggaacactcatttgagcaagaacttgacagcggaggattt

SEQ ID NO: 75 - DNA, 78 bp, Artificial Sequence
  Synthetic primer
aaatccgagcaagaaagtgaaaatggagaaaattatagaaatactcatttgagcaaggagtttggcatcggaggttat

SEQ ID NO: 76 - DNA, 279 bp, Arabidopsis thaliana
aagtttctttttcttttgcaggttaccaatcactctccatggaatactcatttgagcaagatgttggcatgaaattgccgtggtgaaggacgagctgactctatatcgatggtgcctcgacctcgatccttcacctatttattgaaagcatagtttgaatctcattttcaactatgtataaattaatataaacaatgcaggtctagaaaataagctctttttttttcattaccattgactgcctaaaatagtgatgtataactacttttttacatgtta

SEQ ID NO: 77 - DNA, 301 bp, Arabidopsis thaliana
augauucacucacucguauggguggguuggguuuucucuucaugagaugcucauuugagcaagcaauguuuggaagugggacuauuuaauaucauguaugaguuuuaucuuuugttataagaaaaaaagttgggatttcattttcgtgcccacttcacgtgaatctaatgccttcaagtttgtggtctaccttattttctcttatggatacatgtcaatttacgtgtcgtatgagtcaagcctttaatcatcccaacaccatgcacgtcttctagatttgttttcgaccacgattccacca

SEQ ID NO: 78-79 - skipped (2 entries)

SEQ ID NO: 80 - DNA, 110 bp, Artificial Sequence
  Synthetic primer
tctccatggaatactcatttgagcaagatgttggcatgaaattgccgtggtgaaggacgagctgactctatatcgatggtgcctcgacctcgatccttcacctatttatt

SEQ ID NO: 81 - DNA, 110 bp, Artificial Sequence
  Synthetic primer
agatcatggaatactcacttgagcaagaaattataagcacctcgtgttttgcaaagactgcgaggtttctttagttaaaagcctcggccttggaccttcaccatggccaa

SEQ ID NO: 82 - DNA, 110 bp, Artificial Sequence
  Syntetic primer
aagttttggaatactcatttgagcaagaaaatgtggatgctcctggctttgcagggacgacgaggtttgatggttaaggtgcctctacctcggaccttcaccatggccac

SEQ ID NO: 83 - DNA, 110 bp, Artificial Sequence
  Synthetic primer
ttgatgtggaacactcatttgagcaagaaaccaagagccacttctgccttccaaggacgacggtagtgagctgttgaagagcctcgacctcggaccttcatgggggggct

SEQ ID NO: 84 - DNA, 13 bp, Artificial Sequence
  Synthetic primer
  misc_feature (5)..(5): n is a, c, g, or t
  misc_feature (10)..(11): n is a, c, g, or t
ctacnccacnngt